(19) United States
(12) Patent Application Publication
(10) Pub. No.: US 2022/0152251 A1
(43) Pub. Date: May 19, 2022

(54) Non-Aqueous PS/ICP Systems And Application To Surfaces

(71) Applicant: Mi2 Holdings LLC, Houston, TX (US)

(72) Inventors: Andrew Hopkins, Houston, TX (US); Thomas Hopkins, Houston, TX (US)

(73) Assignee: Mi2 Holdings LLC, Houston, TX (US)

(21) Appl. No.: 17/531,527

(22) Filed: Nov. 19, 2021

Related U.S. Application Data
(60) Provisional application No. 63/116,129, filed on Nov. 19, 2020.

(51) Int. Cl. A61L 2/232; A61L 2/10; A61L 2/08; B65D 65/42

(52) U.S. Cl. CPC A61L 2/232; A61L 2/10; A61L 2202/23; B65D 65/42; A61L 2202/16; A61L 2/088

(57) ABSTRACT

A shipping container having photodynamic pathogen reduction properties. The shipping container include photosensitizers that when exposed to light actively and continuously disinfect their surfaces. Methods of adding or incorporating into or on to photosensitizers to a shipping container.

### BACKGROUND

The present disclosures relate generally to coatings and additives for use with, on and in, packaging materials, boxes, containers and shipping materials to provide such materials with photoactive capabilities. These materials create reactive oxygen species (ROS) when exposed to light making the materials actively anti-pathogenic, i.e., an active anti-pathogenic material.

As used herein, unless expressly stated otherwise the term “shipping material” should be given its broadest possible meaning and would include boxes, tubes, containers, carboys, pouches, bags, plastic materials, non-woven materials, fabric materials, woven materials, paper materials, paper board materials, corrugated materials, metal materials, glass materials, composites (including composites of one or more of the foregoing), paper boxes, paper drums, cardboard boxes, plastic containers, plastic boxes, paper boxes, composite boxes, corrugate boxes, pouches, bags, composites of paper fibers and synthetic fibers, as well as, inserts, wrappings and covers for products and produce that are shipped or transferred. The shipping material is made from a structural material, which can be rigid, semi rigid or flexible. The structure material can include or be selected from materials including paper, cardboard, cellulosic materials, paper board, plastics, plastic materials, non-woven materials, fabrics, woven materials, paper materials, paper board materials, corrugated materials, metal materials, glass materials, and composites, including composites of the foregoing, and other materials.

As used herein, unless expressly stated otherwise, “liner”, “liner board”, “liner material” and similar such terms should be given their broadest possible meaning as used in the paper making and box making arts, and would include paper products and board that are used to make boxes, e.g., corrugated boxes, and typically constitute the outer surfaces of the stock used to make a box.

As used herein, unless expressly stated otherwise, “medium”, “corrugation material”, and similar such terms should be given their broadest possible meaning as used in the paper making and box making arts, and would include paper products that are used to make boxes, e.g., corrugated boxes, and typically constitute the inner fluted, folded or corrugated material located on liner material, or located between two outer layers of liner material.

As used herein, unless expressly stated otherwise, the term “shipping container” should be given its broadest possible meaning, and would include boxes, packages, pouches, flexible pouches, bags, tubes, wrappings and containers used by shippers and sellers of goods, such as Amazon, UPS, FedEx and other retainers to package a product or produce for shipment to a consumer or purchaser. The term “shipping container” container covers both business-to-business transfers or shipments and business-to-consumer transfers or shipments, as well as personal shipments, e.g., consumer-to-consumer transfers or shipments.

As used herein, unless expressly stated otherwise the term “pathogen” should be given its broadest possible means in would include any organism that can cause a disease or condition in animals (including humans, pets and livestock) or plants. Pathogens would include, for example, viruses, bacteria, fungi, molds, and parasites. Pathogens would include, for example, among others influenza viruses, corona viruses, SARS-CoV-2 (causing COVID-19), Ebola, HIV, SARS, H1N1 and MRSA, as well as, Campylobacter, Clostridium Perfringens, E. coli, Listeria, Norovirus, Salmonella, Bacillus cereus, Botulism, Hepatitis A, Shigella, Staphylococcus aureus, Staphylococcal (Staph), Vibrio Species Causing Vibriosis, and malaria parasite.

As used herein, unless expressly stated otherwise, the term “fabric” should be given its broadest meaning, and would include natural materials, synthetic materials, woven materials, non-woven materials, as well as, furs and leather.

As used herein, unless expressly stated otherwise, the terms “woven”, “woven fabric”, and “woven material” and similar such terms should be given their broadest meaning, and would include any textile or material that is formed by weaving, that is made on a loom, that has an interlaced pattern of multiple threads including treads at right angles to each other, and that is made of may treads in a pattern having a warp and a weft. Wovens can be made from natural threads, synthetic threads and combinations of these.

As used herein, unless expressly stated otherwise, the terms “nonwoven”, “nonwoven fabric” and “nonwoven material”, and similar such terms, should be given their broadest meanings and would include web structures bonded together by entangling fibers mechanically, thermally fusing the fibers or chemically bonding the fibers, and would include any a sheet, web, or bat of natural man-made and both, fibers or filaments, that are bonded to each other by any of several techniques, including for example, needle punching, stitch bonding, thermal bonding, chemical bonding, hydro entanglement, to name a few. Nonwovens would include staple nonwovens, melt-blown nonwovens, spunlaid nonwovens, spunbond nonwovens, flash spun nonwovens, and air-laid nonwovens, to name few. Nonwovens can be made from natural fibers or materials, synthetic fibers or materials, and combinations of these.

As used herein, unless expressly stated otherwise, “UV”, “ultra violet”, “UV spectrum”, and “UV portion of the spectrum” and similar terms, should be given their broadest meaning, and would include light in the wavelengths of from about 10 nm to about 400 nm, and from 10 nm to 400 nm.

As used herein, unless expressly stated otherwise, the terms “visible”, “visible spectrum”, and “visible portion of the spectrum” and similar terms, should be given their broadest meaning, and would include light in the wavelengths of from about 380 nm to about 750 nm, and 400 nm to 700 nm.

As used herein, unless expressly stated otherwise, the terms “blue”, “blue spectrum”, and “blue portion of the spectrum” should be given their broadest meaning, and would include light having a wavelength from about 400 nm to about 500 nm. Typical blue lasers have wavelengths in the range of 405 nm-495 nm, and about 405 to about 495 nm.

As used herein, unless expressly stated otherwise, the terms “green”, “green spectrum” and “green portion of the spectrum” should be given their broadest meaning, and would include light having a wavelength from about 500 nm to about 575 nm, and from 500 nm to 575 nm.

As used herein, unless stated otherwise, room temperature is 25° C. And, standard ambient temperature and pressure is 25° C. and 1 atmosphere. Unless expressly stated otherwise all tests, test results, physical properties, and values that are temperature dependent, pressure dependent, or both, are provided at standard ambient temperature and pressure, this would include viscosities.

Generally, the term “about” and the symbol “˜” as used herein unless stated otherwise is meant to encompass a variance or range of ±10%, the experimental or instrument error associated with obtaining the stated value, and preferably the larger of these.

As used herein, unless specified otherwise, the recitation of ranges of values, a range, from about “x” to about “y”, and similar such terms and quantifications, serve as merely shorthand methods of referring individually to separate values within the range. Thus, they include each item, feature, value, amount or quantity falling within that range. As used herein, unless specified otherwise, each and all individual points within a range are incorporated into this specification, and are a part of this specification, as if they were individually recited herein.

As used herein, unless expressly stated otherwise terms such as “at least”, “greater than”, also mean “not less than”, i.e., such terms exclude lower values unless expressly stated otherwise.

The terms “photodynamic pathogen reduction”, “PPR” and similar such terms, unless expressly stated otherwise, are to be given their broadest possible meaning and would include a method for ablating, (e.g., killing, destroying, rendering inert), pathogens, including pathogenetic biological tissue, by photo-oxidation utilizing photosensitizer (“PS”) molecules. When the photosensitizer is exposed to a specific wavelength or wavelengths of light, it produces a form of oxygen from adjacent (e.g., in situ, local, intercellular, intracellular) oxygen sources, that kills nearby pathogens, e.g., reactive oxygen species (“ROS”), which includes any form of oxygen that are cyto-toxic to cells or kills or renders inert any pathogen. It being understood that while light across all wavelengths, e.g., UV to visible to IR, is generally used as the activator of the PS, PS typically have a wavelength, or wavelengths where their absorption is highest.

The terms “kill”, “killing” and similar such terms, unless expressly stated otherwise, when used in context of a pathogen, including a virus, should be given its broadest possible meaning, and would include rendering the pathogen inactive, so that it cannot infect, or harm, an animal, including a manual or human.

The terms “active anti-pathogen”, “active surface”, “active material” “photoactive surface” and “photoactive material”, and “photoactive” and similar such terms, unless expressly stated otherwise, should be given their broadest possible meaning and would include any material or surface, as well as agents that are triggered to product active oxygen, such as a reactive oxygen species (“ROS”) or other active therapeutic materials, when exposed to energy sources including energy sources other than light, as activators. These would include materials or agents that are activated by energy sources such as radio waves, other electromagnet radiation, magnetism, and sonic (e.g., Sonodynamic therapy or SDT).

The terms “photosensitizer” and “PS” and “photoactive agent” and similar such terms, unless expressly stated otherwise, should be given their broadest possible meaning and would include any dye, molecule or modality that when exposed to light produces, or causes the production of, ROS, or other active agents that are cyto-toxic to cells, kill tissue, ablates tissue, destroys tissue or renders a pathogen inert (i.e., pathogenic).

As used herein, unless expressly stated otherwise, “photosensitizer-inclusion complex former”, “PS-ICF”, “ICF-PS” and similar such terms include all compositions and formulations having at least a photosensitizer (PS) associated with an inclusion complex former (ICF), including with and without a nanoparticle, with and without a targeting agent, with and without a nanoparticle or targeting agent, and with and without a nanoparticle and targeting agent.

As used herein, unless expressly stated otherwise,

As used herein, unless expressly stated otherwise, “SARS-CoV-2”, “COVID 19”, “Covid-19”, “Covid Contamination”, and similar such terms should be given their broadest possible meaning and would include any virus or pathogen that causes COVID-19 or causes any symptoms, diseases or conditions presently or in the future associated with COVID-19, as well all mutations and variations of the SARS-CoV-2 virus.

COVID-19, which is caused by SARS-CoV-2 virus is a devastating, highly contagious virus that spreads via airborne transmission (e.g., coughing and sneezing) and surface contact. The challenges in preventing this spread are is its ease of transference because of its ability of the virus to survive on surfaces (including PPE) for extended periods of time. Hand sanitizer, soap and bleach-based products, these products lack the ability to provide lasting disinfection of the virus, they begin sterile but can quickly become contaminated and transfer live virus. Instead, these products only provide a one-time cleanse. Post-cleanse, these surfaces are susceptible to future contamination, which contributes to the rapid spread of the virus, even with the nationwide shelter in place order.

Covid-19 (SARS-CoV-2) is a highly infectious disease with potentially severe outcomes. Beyond the obvious human to human transmission pathway, the virus has been shown to be viable for many hours or days on contaminated surfaces, providing a major secondary route for continued transmission. This problem exists as well for other pathogens.

This Background of the Disclosure section is intended to introduce various aspects of the art, which may be associated with embodiments of the present disclosures. Thus, the forgoing discussion in this section provides a framework for better understanding the present disclosures, and is not to be viewed as an admission of prior art.

### SUMMARY

There has been a long-standing and unfulfilled need for shipping materials to address the risks and harm from pathogens to packers, shippers and persons receiving packages. In particular, there is a critical and urgent need to address the risks and harms from the SARS-CoV-2 virus and COVID-19, in the shipping and receiving of packages and the goods they contain.

The present disclosures, among other things, solve these needs by providing the photoactive compositions, materials, articles of manufacture, devices, methods and processes taught, disclosed and claimed herein.

### BRIEF DESCRIPTION OF THE DRAWINGS

FIGS. 1 to 4 are schematics of a paper making machine and process for making embodiments of the active materials in in accordance with the present disclosures.

FIG. 5 is a perspective view of shipping containers, e.g., boxes having active surfaces in accordance with the present disclosures.

FIG. 6 is a perspective of structural materials used to make shipping containers having active surfaces in accordance with the present disclosures.

FIG. 7 is a plan view of an unassembled container showing a top active surface in accordance with the present disclosures.

FIG. 8 is a perspective view of an insert having active surface for use in containers having active surfaces in accordance with the present disclosures.

FIG. 9 is schematic illustration of a configuration for a nanocomposition in accordance with the present disclosure.

FIG. 10 is a schematic illustration of a configuration for a nanocomposition in accordance with the present disclosure.

FIG. 11 is a schematic illustration of a linkers and end group conversion in accordance with the present disclosure.

FIG. 12 is a schematic illustration of a configuration for a nanocomposition in accordance with the present disclosure.

FIG. 13A is a schematic illustration of a synthesis method for a nanocomposition in accordance with the present disclosure.

FIG. 13B is a schematic illustration of a synthesis method for a nanocomposition in accordance with the present disclosure.

FIG. 14A is a schematic illustration of a synthesis method for a nanocomposition in accordance with the present disclosure.

FIG. 14B is a schematic illustration of a synthesis method for a nanocomposition in accordance with the present disclosure.

FIG. 15 is a schematic illustration of a configuration for a targeted nanocomposition in accordance with the present disclosure.

FIG. 16 is a schematic illustration of a configuration for a targeted nanocomposition in accordance with the present disclosure.

FIG. 17 is a schematic representation of the production of ROS

FIG. 18 shows examples of photosensitizers for use in nanocomposition in accordance with the present disclosure.

FIG. 19 is a schematic representation of a photodynamic disinfection processes in accordance with the present disclosure.

### DESCRIPTION OF THE PREFERRED EMBODIMENTS

The present disclosures relate to the use of photosensitizers to provide photoactive materials and surfaces for containers, boxes, and shipping materials, to reduce, mitigate, block and kill or render inert pathogens.

In general, embodiments of the present disclosures are shipping materials having PPR properties.

In general, embodiments of the present disclosures are shipping materials that have active anti-pathogen properties and surfaces.

In general, embodiments of the present disclosures are shipping materials having a PS on one or more of their surfaces, and incorporated with in the material itself.

In general, embodiments of the present disclosure are methods of coating shipping materials with a composition having a PS, so PS is provided to the surface of the shipping material, imparting PPR properties to the shipping material.

In general, embodiments of the present disclosure are methods of incorporating a PS into, (e.g., within, throughout, on the surface) the stock material used to make shipping materials, imparting PPR properties to the shipping materials.

In general, embodiments of the present disclosures are shipping materials and may also have a PS including within such materials, methods and uses that have a photosensitizer, a photosensitizer and an inclusion complex former, a nanocomposition and other combinations of these. The present disclosures provide containers, boxes, and shipping materials, that are active pathogen barriers, and are photoactive materials.

The containers, boxes, and shipping materials, can have the photosensitizer applied to the surface of the materials used to make the containers, boxes, and shipping materials. Thus, the photosensitizer can be added during the paper making process. Recognizing that the ambient light present during and after the application of the photosensitizer should not activate the photosensitizer.

The photosensitizer (PS), PS compositions, and PS formulations for adding or delivering a PS to a shipping material or structural material (e.g., flat stock, raw stock material or structural material for making a shipping material) can be any one or more of the PS, PS compositions, and PS formulations disclosed and taught in: (i) U.S. Provisional Patent Application Ser. No. 63/023,807 the entire disclosure of which is incorporated herein by reference and with is attached as Appendix A and forms a part of this Specification; and (ii) Appendix B which is attached hereto and forms a part of this Specification.

The photosensitizers can be added to the paper or board as it is being made, and thus be on the surface and throughout the board. This provides the advantage of open end or edges of the box also having antipathogenic capabilities.

The photosensitizers (“PS”) can be added to the paper or board after it has been made, in either the lay flat configuration or the assembled configuration.

As there are literally any number of PS's that we can use the water temp should not be an issue, nor should a pH from 3-11 (there are many that are ok outside this range too)

A material comprising paper fibers, selected from the group of liner board, liner, medium, corrugated, corrugated containers, boxes, corrugated boxes, sheet material, and corrugated sheet material having a surface having a complex or composition of a photosensitizer for use in forming active surfaces and active materials to kill or render inactive pathogens. Treating a material, comprising paper fibers, selected from the group of liner board, liner, medium, corrugated, corrugated containers, boxes, corrugated boxes, sheet material, and corrugated sheet material whereby treated material is an active surface that kills or renders inactive pathogens upon illumination with sufficient light.

### EXAMPLES

The following examples are provided to illustrate various embodiments of systems, processes, compositions, applications and materials of the present disclosures. These examples are for illustrative purposes, may be prophetic, and should not be viewed as, and do not otherwise limit the scope of the present disclosures.

### Example 1

About 1-2 μg of PS, e.g., Methylene Blue (MB)/cm2 or 10 to 20 mg/m² for MB—which has a mwt ˜320, so this equates to 3×10-5 to 6×10-5 mols per m² (or 3-6 nmols(nanomoles)/cm²)

### Example 2

1 nmols-100 nmols/cm² of PS, smaller and large amounts of PS can be used per surface area of the container, e.g., box.

### Example 3

The PS is added into the liquid paper slurry in the paper machine (thin stock or thick stock), or white water of the paper making process. In this manner the PS is including within and throughout the paper, as well as on its surfaces and edges.

### Example 4

The PS is applied to the surface of the paper during the paper making process at the presses, size press, dryers, calendars, by spraying on the paper wed (at any moisture content of the web) by foam coating, and at the reel as the paper web is wound.

### Example 5

The PS is applied to the stock, e.g. paper board, cardboard, flat stock, as it is converted (e.g., folded and glued) into a container, e.g., a box.

### Example 6

The PS could also be post coated in the following types of formulation

As the PS alone in a suitable solvent (water, buffered to pH 3-11, alcohol or any other suitable solvent)

As the PS and the ICF (inclusion complex former)

As the PS and ICF in a coating formulation

Water or organic solvent based—the following solids

With a coating polymer (PVA, PVAc, Polyoxazoline, Polyvinyl pyrrolidone, cellulose acetate, carboxy methyl cellulose)—or other suitable coating polymer)—typically in the 0.1-5% range

With a film former (for spreading)—Typically <1%

With a soap/surfactant—typically <1%

With a secondary antimicrobial—eg a QAC or Quat (same thing)—a cationic surfactant, typically 0.1-5%

A buffer if required—typically 1 mMolar to 100 mMolar

All of this to deliver a final PS conc in the 1-2 μg/cm² concentration.

### Example 7

Articles treated to provide an active and prolonged surface disinfection to single use/limited use container, e.g., box, package, shipping material.

This refers to the in-situ treatment/production of articles such that:

Said articles when exposed to a suitable light source (daylight or ambient lighting) actively and continuously disinfect their (own) surface and other articles placed close to them

But may also retain anti-microbial activity in the dark through a combination of other anti-microbial agents

Certain anti-microbials may have a synergistic effect, where PS (ROS)+antimicrobial action is more than 1+1=2

QAC's and active oxygen species appear act synergistically in the breakdown of biofilms and certain pathogens

Protect the handler/user from infection by pathogens transmitted via contact with said surface

These may be applied to one time/limited time use product

Said items may be recycled into the usual stream without the need for separation or special treatment

Said items provide for a continually disinfected surface for the lifetime of use

is applicable for items made with a wide variety of materials, not limited to but including papers, cardboards, wood, metal, plastic (all types), ceramics, glass and composites of all the above.

Examples for use may be

Packaging materials—boxes, wrapping paper or plastic

Plastic films or bags for the protection of other products

Mats, coverings etc to protect work surfaces, tables etc

Through the “adjacent disinfection mechanism” of ROS items may also provide temporary but effective anti-microbial protection to items place on the surface of such items—silverware, phones, keys, coins, surgical items (in healthcare environment)

### Example 8

A range of formulations—optimized for the surface in question—that when applied provide for a coating that in the presence of light (daylight or ambient) continuously generate an effective flux of reactive oxygen species that inactivate substantially all pathogens present on, or in close proximity to the surface

Formulation Contains

A photosensitizer or mixture of photosensitizers—drawn from any class of Photosensitizers that produce ROS in the presence of light

A carrier liquid (may be aqueous or non-aqueous or mixture of)—that dissolves or usefully disperses all ingredients

An inclusion forming complexing agent capable of forming said inclusion complex with a photosensitizer or mixture of photosensitizer

In addition, formulation may contain

A soluble or dispersible polymer including, but not limited to; Polyvinyl alcohol, polyvinyl acetate, polyester, polyvinyl pyrrolidone, polyoxazoline etc.

A dispersant of mixtures thereof

A surfactant or mixtures thereof, cationic, anionic, or non-ionic

Other anti-microbial agents

Quaternary ammonium cationic surfactants (QAC's or Quats)

Pesticides

A buffer or mixture of buffers to regulate pH

A nanoparticle conjugates to the Photosensitizer

An odor reducing material

A scent

Colorants

Film formers

Any other material used in the formation of coatings

Formulations are applied either during the manufacturing process of the article or at point of use

Formulation is applied through standard processes including but not limited to Spraying

Rolling

Flooding (doctor blade)

Brushing

Laminating

Painting

Aerosol

Is applied to deliver an effective concentration of “Active” PS to the surface (addition of the inclusion complex former optimizes this by deliver more of the ‘active monomeric’ form of the photosensitizer to the surface

### Example 9A

Coated Rolls of paper (one or two sides) used to cover, laminate, wrap items, or as “Stand-alone” product to protect a surface (eg mats)

### Example 9B

Coated rolls of cardboard stock (one or two sides) for use in cartons or corrugated boxes or other corrugated products

### Example 9C

Coated rolls of solid plastic (polyethylene, polypropylene, polyamide, polyester . . . ) film (one or two sides) for use to laminate/cover other materials (eg cardboard, paper, work surfaces, ceramics, metals and composites thereof.)

### Example 9D

Coated plastic foamed roll/sheet products (eg polystyrene, poly olefins)—cut to size and used as covering and mats

### Example 9E

Metal cans for packaging

### Example 9F

Concentrates, finished formulations in tanks, bottles, spray cans—to apply coating at point of use—ie to an untreated cardboard boxes, metal containers—applied through any common coating process.

### Example 9G

Boxes with PS surfaces

### Example 9H

Work surfaces with PS surfaces

### Example 9I

Healthcare work surfaces/surgical theaters with PS surfaces

### Example 9J

Paper coverings with PS surfaces

### Example 9K

Metal cans with PS surfaces

### Example 10

The embodiments of Examples 9A to 9K having PPR surfaces and properties.

### Example 11

The embodiment of Examples 9A to 9K that are active anti-pathogen materials and surfaces.

The present inventions relate to the use of photosensitizers to provide photoactive materials and surface to reduce, mitigate, block and kill or render inert pathogens. In particular, the present inventions relate to such materials, methods and uses that have a photosensitizer and an inclusion complex former, a nanocomposition and both. The present inventions provide surfaces and materials that are active pathogen barriers, and active anti-pathogens.

Embodiments of the present inventions relate generally to photodynamic applications, additives, coatings and compositions including nanocompositions, both non-targeted and targeted, and uses of these in active, e.g., dynamic, anti-pathogenic materials and methods; such as for treating, managing, blocking, reducing and eliminating pathogens in, and on, surfaces, fabrics, products, face masks, gloves, head coverings, shoe coverings, non-woven materials, paper materials, countertops, packaging, equipment, medical equipment, personal protective equipment (PPE). In particular, in an embodiment, the present inventions relate to materials and composition that upon exposure to light actively remove, reduce and eliminate pathogens that are in contact with such materials and compositions.

Viruses have been estimated to be the most abundant and diverse biological systems on earth. Size typically ranges from 20-300 nm. Viruses depend on living cells for their reproduction and are classified according to their genome and method of reproduction (Baltimore classification). They may consist of a DNA or RNA (single or double stranded) core an outer protein cover and in some virus classes, lipids.

In general embodiments of the present inventions relate to formulations that generally include an inclusion complex former (“ICF”) and a PS. These ICF-PS embodiments may also include, or be based upon, an NP, and a TA and NP. ICFs would include, for example, cyclodextrins (including all derivatives thereof, as well as alpha/beta/gamma and their derivatives), calixarenes, cryptands and crown ethers.

Generally, embodiments of cyclodextrins for use as an ICF in the present formulations include hydrophobic, hydrophilic, polymeric, ionized, non-ionized, and many other derivatives of cyclodextrins. In general, derivatization of cyclodextrin proceeds via a reaction in which the OH group at position 2, 3, and/or 6 of the amylose ring of cyclodextrin is replaced with a substituent. The substituents include neutral functional groups, anionic functional groups, cationic functional groups, and combinations of these.

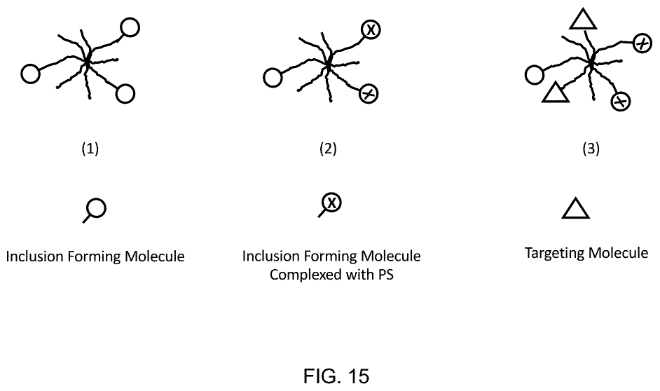

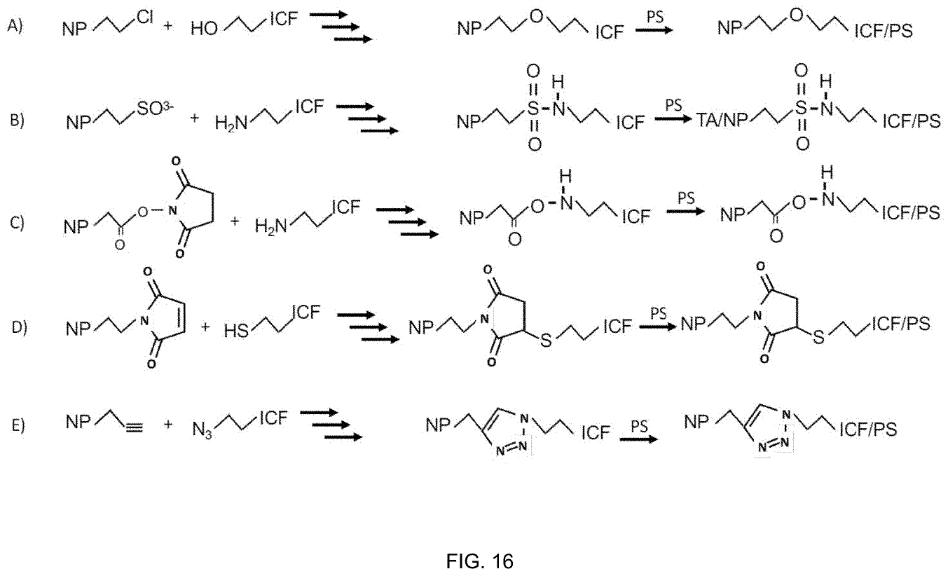

Examples of ICF-nanocompositions, and both non-targeted and targeted ICF-NP-PS are shown in FIGS. 15 and 16.

Cyclodextrin derivatives, include for example, such as alkylated cyclodextrins include sulfoalkyl ether cyclodextrins, alkyl ether cyclodextrins (eg, methyl, ethyl and propyl ether cyclodextrins), hydroxyalkyl cyclodextrins, thioalkyl ether cyclodextrins, carboxylated cyclo cyclodextrins, dextrin (eg, succinyl-β-cyclodextrin and the like), sulfated cyclodextrin and the like, but not limited thereto. Also included are alkylated cyclodextrins having two or more functional groups such as sulfoalkyl ether-alkyl ether-cyclodextrins (for example, WO 2005/042584, which is incorporated herein in its entirety by reference) and US Patent Application Publication No. 2009/0012042. In particular, alkylated cyclodextrins having a 2-hydroxypropyl group, a sulfoalkyl ether group and both are provided for example. Sulfobutylether derivatives of β-cyclodextrin (“SBE-β-CD”) are commercialized by CyDex Pharmaceuticals, Inc. as CAPTISOL R and ADVASEP®. The anionic sulfobutyl ether substituent improves the water solubility and safety of the parent β-cyclodextrin, which can reversibly form a complex with the active pharmaceutical agent, whereby the solubility of the active pharmaceutical agent. And in some cases increase the stability of the active pharmaceutical agent in aqueous solution. CAPTISOL R has the chemical structure of Formula X

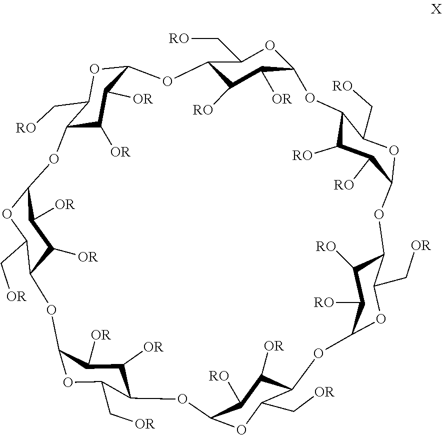

In the formula, R is (—H)²¹⁻ⁿ or ((—CH₂)₄—SO₃⁻ Na⁺)ₙ, and n is 6 to 7.1. Sulfoalkylether-derivatized cyclodextrins (such as CAPTISOL®) are all incorporated herein by reference in their entirety to U.S. Pat. Nos. 5,134,127 and 5,376,645. And, for example, using the batch method described in U.S. Pat. No. 6,153,746.

Examples of structures for cyclodextrins include:

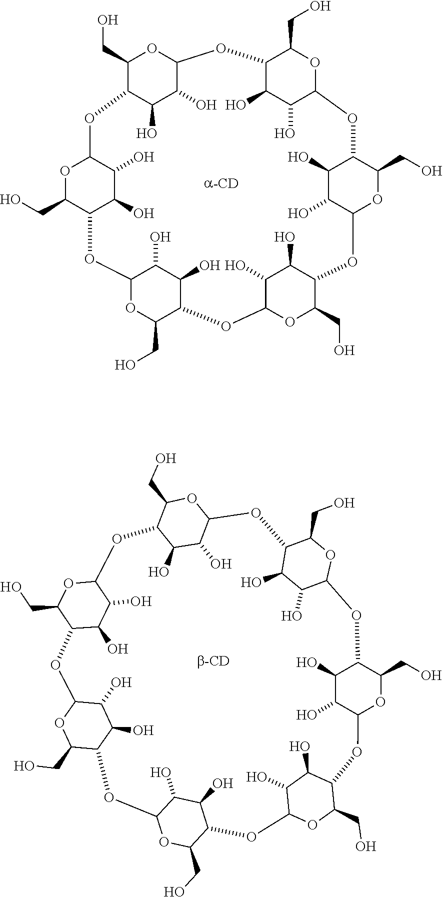

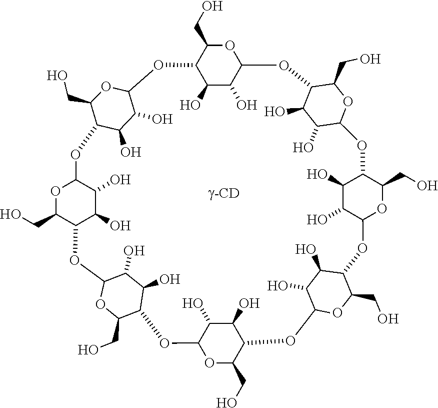

Cyclodextrins can be made from the cyclomaltodextrin glucanotransferase (E.C. 2.4.1.19; CGTase) catalyzed degradation of starch. They form soluble inclusion compounds with less-hydrophilic molecules that fit into their cavities. Generally, there are three common cyclodextrins with 6, 7 or 8 D-glucopyranosyl residues (α-(alpha), (β-(beta), and γ-(gamma) cyclodextrin respectively) linked in a ring by α-1,4 glycosidic bonds. The glucose residues have the ⁴C₁ (chair) conformation. All three cyclodextrins have similar structures (that is, bond lengths and orientations) apart from the structural necessities of accommodating a different number of glucose residues. They can be viewed as presenting a bottomless bowl-shaped (truncated cone) molecule stiffened by hydrogen-bonding between the 3-OH and 2-OH groups around the outer rim. The hydrogen bond strengths are α-cyclodextrin β-cyclodextrin and γ-cyclodextrin.

The flexible 6-OH hydroxyl groups are also capable of forming linking hydrogen bonds around the bottom rim, but these are destabilized by dipolar effects, easily dissociated in aqueous solution and not typically found in cyclodextrin crystals. The hydrogen bonding is all 3-OH (donor) and 2-OH (acceptor) in α-cyclodextrin but flips between this and all 3-OH (acceptor) and 2-OH (donor) in β- and γ-cyclodextrins [918].

Cyclodextrin Shape

The cavities have different diameters dependent on the number of glucose units (empty diameters between anomeric oxygen atoms given in the diagram below). The side rim depth (shown below in the diagrams) is the same for all three (at about 0.8 nm).

| | | Outer | | | Cavity | | | |
|---|---|---|---|---|---|---|---|---|
| | | diameter, | Cavity diameter (nm) | | volume, | Solubility, | Hydrate H₂O | |
| Cyclodextrin | Mass | (nm) | Inner rim | Outer rim | (mL/g) | g/kg H₂O | cavity | external |
| α, (glucose)₆ | 972 | 1.52 | 0.45 | 0.53 | 0.10 | 129.5 | 2.0 | 4.4 |
| β, (glucose)₇ | 1134 | 1.66 | 0.60 | 0.65 | 0.14 | 18.4 | 6.0 | 3.6 |
| γ, (glucose)₈ | 1296 | 1.77 | 0.75 | 0.85 | 0.20 | 249.2 | 8.8 | 5.4 |

Impurities present in the alkylated cyclodextrin composition may reduce the shelf life and potency of the active drug composition. Impurities can be removed from the alkylated cyclodextrin composition by exposure to activated carbon (eg, mixing with activated carbon). The treatment of cyclodextrin-containing aqueous solutions and aqueous suspensions with activated carbon is known. See, for example, U.S. Pat. Nos. 4,738,923, 5,393,880, and 5,569,756, the entire disclosures of each of which are incorporated by reference.

As used herein, an embodiment of cyclodextrins for use in the formulation as an ICF, includes any of the known cyclodextrins such as unsubstituted cyclodextrins containing from six to twelve glucose units, especially, alpha-cyclodextrin, beta-cyclodextrin, gamma-cyclodextrin and/or their derivatives and/or mixtures thereof. The alpha-cyclodextrin consists of six glucose units, the beta-cyclodextrin consists of seven glucose units, and the gamma-cyclodextrin consists of eight glucose units arranged in donut-shaped rings. The specific coupling and conformation of the glucose units give the cyclodextrins a rigid, conical molecular structures with hollow interiors of specific volumes. The “lining” of each internal cavity is formed by hydrogen atoms and glycosidic bridging oxygen atoms therefore, this surface is fairly hydrophobic. The unique shape and physical-chemical properties of the cavity enable the cyclodextrin molecules to absorb (form inclusion complexes with) organic molecules or parts of organic molecules which can fit into the cavity. Many odorous molecules can fit into the cavity including many malodorous molecules and perfume molecules. Therefore, cyclodextrins, and especially mixtures of cyclodextrins with different size cavities, can be used to control odors caused by a broad spectrum of organic odoriferous materials, which may, or may not, contain reactive functional groups. The complexation between cyclodextrin and odorous molecules occurs rapidly in the presence of water. However, the extent of the complex formation also depends on the polarity of the absorbed molecules. In an aqueous solution, strongly hydrophilic molecules (those which are highly water-soluble) are only partially absorbed, if at D. Therefore, cyclodextrin does not complex effectively with some very low molecular weight organic amines and acids when they are present at low levels on fabrics, e.g. as the composition dries on the treated fabrics. As the water is being removed however, e.g., water is being extracted from carpet by a carpet extractor, some low molecular weight organic amines and acids have more affinity and will complex with the cyclodextrins more readily.

The cavities within the cyclodextrin in the stable, aqueous composition of the present invention should remain essentially unfilled (the cyclodextrin remains uncomplexed) while in solution, in order to allow the cyciodextrin to absorb various odor molecules when the solution is applied to a surface. Non-derivatised (normal) beta-cyciodextrin can be present at a level up to its solubility limit of about 1.85% (about 1.85 g in 100 grams of water) wider the conditions of use at room temperature.

Preferably, the cyclodextrin used in the present invention is highly water-soluble such as, alpha-cyclodextrin and/or derivatives thereof, gamma-cyclodextrin and/or derivatives thereof, derivatised beta-cyclodextrins, and/or mixtures thereof. The derivatives of cvclodextrin consist mainly of molecules wherein some of the OH groups are converted to OR groups. Cyclodextrin derivatives include, e.g., those with short chain alkyl groups such as methylated cyclodextrins, and ethylated cyclodextrins, wherein R is a methyl or an ethyl group; those with hydroxyalkyl substituted groups, such as hydroxypropyl, cyclodextrins and/or hydroxyethyl cyclodextrins, wherein R is a —CH₂—CH(OH)—CH₃ or a ⁻CH₂CH₂—OH group; branched cyclodextrins such as maltose-bonded cyclodextrins; cationic cyclodextrins such as those containing 2-hydroxy-3-(dimethylamino)propyl ether, wherein R is CH₂—CH(OH)—CH₂—N(CH₃)₂ which is cationic at low pH; quaternary ammonium, e.g., 2-hydroxy-3-(trimethylammonio)propyl ether chloride groups, wherein R is CH₂—CH(OH)—CH₂—N⁺(CH₃)₃Cl⁻; anionic cyclodextrins such as carboxymethyl cyclodextrins, cyciodextrin sulfates, and cvclodextrin succinylates; amphoteric cyclodextrins such as carboxymethyl/quaternary ammonium cyclodextrins; cyclodextrins wherein at least one glucopyranose unit has a 3-6-anhydro-cyclomalto structure, e.g., the mono-3-6-anhydrocyclodextrins, as disclosed in “Optimal Performances with Minimal Chemical Modification of Cyclodextrins”, F. Diedaini-Pilard and B. Perly, The 7th International Cyclodextrin Symposium Abstracts, April 1994, p. 49, said references being incorporated herein by reference; and mixtures thereof. Other cyciodextrin derivatives are disclosed in U.S. Pat. No. 3,426,011, Parmerter et al., issued Feb. 4, 1969; U.S. Pat. Nos. 3,453,257; 3,453,258; 3,453,259; and 3,453,260, all in the names of Parmerter et al., and all issued Jul. 1, 1969; U.S. Pat. No. 3,459,731, Gramera et al., issued Aug. 5, 1969; U.S. Pat. No. 3,353,191, Parmerter et al., issued Jan. 5, 1971; U.S. Pat. No. 3,565,887, Parmerter et al., issued Feb. 23, 1971; U.S. Pat. No. 4,535,152, Szejtli et al., issued Aug. 13, 1985; U.S. Pat. No. 4,616,008, Hirai et al., issued Oct. 7, 1986; U.S. Pat. No. 4,678,598, Ogino et al., issued Jul. 7, 1987; U.S. Pat. No. 4,638,058, Brandt et al., issued Jan. 20, 1987; and U.S. Pat. No. 4,746,734, Tsuchiyama et al., issued May 24, 1988; all of said patents being incorporated herein by reference. Further cyciodextrin derivatives suitable herein include those disclosed in V. T. D'Souza and K. B. Lipkowitz, CHEMICAL REVIEWS: CYCLODEXTRINS, Vol. 98, NO. 5 (American Chemical Society, July/August 1998), the entire disclosures of each of which are incorporated herein by reference.

In embodiments highly water-soluble cyclodextrins are those having water solubility of at least about 10 g in 100 ml of water at room temperature, preferably at least about 20 g in 100 ml of water, more preferably at least about 25 g in 100 ml of water at room temperature can be used in the formulation.

Examples of a water-soluble cyclodextrin derivatives suitable for use herein are hydroxypropyl alpha-cyclodextrin, methylated alpha-cyclodextrin, methylated beta-cyclodextrin, hydroxyethyl beta-cyciodextrin, and hydroxypropyl beta-cyciodextrin. Hydroxyalkyl cyclodextrin derivatives preferably have a degree of substitution of from about 1 to about 14, More preferably from about 1.5 to about 7, wherein the total number of OR groups per cyclodextrin is defined as the degree of substitution. Methylated cyclodextrin derivatives typically have a degree of substitution of from about 1 to about 18, preferably from about 3 to about 16. A known methylated beta-cyclodextrin is, heptakis-2,6-di-O-methyl-β-cyclodextrin, commonly known as DIMEB, in which each glucose unit has about 2 methyl groups with a degree of substitution of about 14. An example of a commercially available, methylated beta-cyclodextrin is a randomly methylated beta-cyclodextrin, commonly known as RAMEB, having different degrees of substitution, normally of about 12.6. RAMEB is more preferred than DIMEB, since DIMEB affects the surface activity of the preferred surfactants more than RAMEB. The preferred cyclodextrins are available, e.g., from Cerestar USA, Inc. and Wacker Chemicals (USA), Inc.

In embodiments a mixture of cyclodextrins are used. Such mixtures absorb odors more broadly by complexing with a wider range of odoriferous molecules having a wider range of molecular sizes. At least a portion of the cyclodextrin is alpha-cyclodextrin and its derivatives thereof, gamma-cyclodextrin and its derivatives thereof, and/or derivatized beta-cyclodextrin, more preferably a mixture of alpha-cyclodextrin, or an alpha-cyclodextrin derivative, and derivatized beta-cyclodextrin, even more preferably a mixture of derivatized alpha-cyclodextrin and derivatized beta-cyclodextrin, most preferably a mixture of hydroxypropyl alpha-cyclodextrin and hydroxypropyl beta-cyclodextrin, and/or a mixture of methylated alpha-cyclodextrin and methylated beta-cyclodextrin.

In an embodiment when dilute compositions are used, the level of cyclodextrin is from about 0.3% to about 50%, more preferably from about 0.5% to about 40%, by weight of the composition. When concentrated compositions are used, the level of cyclodextrin is from about 2% to about 80%, more preferably from about 3% to about 70%, by weight of the concentrated composition.

The present inventions further relate to nanocompositions. In particular, the present inventions provide nanocompositions having a nanoparticle and a PS, for use in coatings, solutions, and materials to make these materials active materials that are anti-pathogenic.

In a preferred embodiment the PS composition upon application and activation with light, does not damage or destroy the treated article, surface or material. Thus, the treatment of articles does not adversely change the material properties of the article, only adding the property of being an active. Anti-pathogen.

An embodiment of the present inventions is a composition having a core molecule, to which a PS is linked (e.g., chemically, covalently or otherwise attached). In preferred embodiments, the photosensitizer is a photoactive dye, and the core molecule is a multi-arm nanoparticle, a linear molecule, PEG, a multi-arm PEG, 8PEG, 8PEGA and 8PEGMAL. These embodiments are used to provide PPR.

An embodiment of the present inventions is a composition having a core molecule, to which a pathogen specific TA and a PS are linked (e.g., chemically, covalently or otherwise attached). In preferred embodiments, the photosensitizer is a phthalocyanine dye, and the core molecule is a multi-arm nanoparticle, a linear molecule, PEG, a multi-arm PEG, 8PEG, 8PEGA and 8PEGMAL. These embodiments is used to provide pathogenic PPR.

The targeting agent (TA) can be an agent e.g., peptide, antibody, protein, or small molecule, that targets a pathogen. As such these targeting agents will be referred to as Pathogen specific targeting agents (PSTA) Pathogen targeting peptides (PTP) in embodiments may be a preferred TA. The TA's, are linked to a nanoparticle to form a nanocomposition that also may have a PS. The TA nanoparticle composition may be used for imaging. The TAs are specific to a particular pathogen, or spices, group of family of pathogens. The TA can bind to, target or be specific for unique identifiers, e.g., structures, on the pathogen. The PSTA nanocomposition is transduced into or otherwise affixed to the pathogen at much higher levels than it is transduced into or affixed to other tissues and cells, such as, for example, red blood cells, liver, kidney, lung, skeletal muscle, cardiac, epithelial or brain. In certain embodiments the ratio of selectivity of PSTA nanocomposition for the pathogen relative to all other tissues and cells present in the patient, is at least 2:1 and greater, is at least 3:1 and greater, is at least 4:1 and greater, is at least 10:1 and greater, and is at least 100:1 and greater.

The photoactive agent can be any dye or molecule that produces, or causes the production of ROS when exposed to light, or produces other compounds when exposed to light that kill, destroy or render inert, the pathogen. Examples of PS include, for example, IR700, methylene blue (MB), chlorin e6 (Ce6), Rose Bengal, Robflavin, and Erythrosine.

An embodiment of the present nanocompositions is a nanoparticle and a PS. This embodiment is used to provide PPR.

An embodiment of the present nanocompositions is a nanoparticle, a phthalocyanine PS, where the phthalocyanine is a phthalocyanine die disclosed and taught in U.S. Pat. No. 7,005,518, and a PSTA. This embodiment is used to provide PPR.

An embodiment of the present nanocompositions is a nanoparticle, where the nanoparticle is PEG, and preferably 8PEGA, a PS. This embodiment is used to provide PPR.

An embodiment of the present nanocompositions is a nanoparticle, where the nanoparticle is PEG, and preferably 8PEGA, a phthalocyanine PS, where the phthalocyanine is a phthalocyanine die disclosed and taught in U.S. Pat. No. 7,005,518, and a PSTA. This embodiment is used to provide PPR.

An embodiment of the present nanocompositions is a nanoparticle, where the nanoparticle is PEG, and preferably 8PEGA, a phthalocyanine PS, where the phthalocyanine is a phthalocyanine die disclosed and taught in U.S. Pat. No. 7,005,518, and a PSTA. This embodiment is used to provide PPR.

As used herein 8PEG refers to, and would include, any 8-arm polyethylene glycol (PEG) molecule (e.g., nanoparticle). 8PEG would include all 8PEGs where one or more of the end groups of the arms is modified. For example, 8PEG would include 8PEGA (8PEG-A, and similar terms) which is 8PEG having amine terminated end groups on the arms (one, two and preferably all arms). For example, 8PEG would include 8PEGMAL (8PEG-MAL and similar terms) which is 8PEG having maleimide terminated end groups on the arms (one, two and preferably all arms). These 8PEGs would include nanoparticles having a hydrodynamic diameter (e.g., size) of 25 nm and less, a hydrodynamic diameter of 10 nm and less, and having a hydrodynamic diameter of from about 30 nm to about 5 nm, and having a hydrodynamic diameter of from about 20 nm to about 5 nm. These 8PEGs would include nanoparticles that are 20 kilodaltons (kDa) and greater, that are 40 kDa and greater, and that are from about 15 kDa to about 50 kDa, and that are from about SkDa to about 100 kDa.

IRDye 700DX HHS Ester (“IR700”) is an example of a photosensitizer for the present embodiments of nanocompositions and for the treatment of pathogen conditions using the present embodiments of the targeted nanoparticle and nanocompositions based photodynamic therapies.

IR700 is a phthalocyanine dye that has minimal sensitive to photobleaching, and is thus preferred to many other organic fluorochromes. IR700 is water soluble, having good solubility. It is salt tolerant, having good salt tolerance. IR700 is available from LI-Cor and is an embodiment disclosed in U.S. Pat. No. 7,005,518, the entire disclosure of which is incorporated herein by reference.

US Patent Publication No. 2015/0328315 teaches and disclose photodynamic therapies, nanocompositions, targeted nanocompositions, imaging and theranostics, the entire disclosure of which is incorporated herein by reference.

The photosensitizer (PS) can be any dye or molecule that produces ROS when exposed to light, or produces other compounds when exposed to light that kill the pathogen. Examples of photoactive agents include, for example, methylene blue (MB), chlorin e6 (Ce6), Rose Bengal, gold.

The PS can be the compositions disclosed and taught in U.S. Pat. Nos. 8,562,944, 8,906,343, and 9,045,488.

The PS can be PHOTOFRIN,

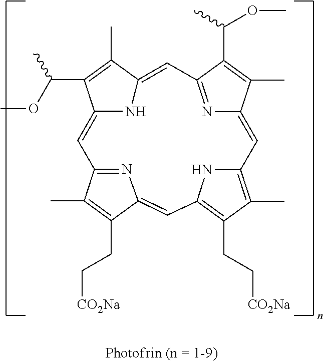

The PS can be Photochlor (CAS #149402-51-7)

The PS can be

| | | WAVELENGTH, |
|---|---|---|
| PHOTOSENSITIZER | STRUCTURE | nm |
| Porfimer sodium (Photofrin) (HPD) | Porphyrin | 630 |
| ALA | Porphyrin | 635 |
| | precursor | |
| ALA esters | Porphyrin | 635 |
| | precursor | |
| Temoporfin (Foscan) (mTHPC) | Chlorine | 652 |
| Verteporfin | Chlorine | 690 |
| HPPH | Chlorin | 665 |
| SnEt2 (Purlytin) | Chlorin | 660 |
| Talaporfin (LS11, MACE, NPe6) | Chlorin | 660 |
| Ce6-PVP (Fotolon), Ce6 derivatives | Chlorin | 660 |
| (Radachlorin, Photodithazine) | | |
| Silicon phthalocyanine (Pc4) | Phthalocyanine | 675 |
| Padoporfin (TOOKAD) | Bacteriochlorin | 762 |
| Motexafin lutetium (Lutex) | Texaphyrin | 732 |

Further the PS for the present nanocompositions can be one or more of the forgoing and one or more of the materials and compositions identified in Redmond, A Compilation of Singlet Oxygen Yields from Biologically Relevant Molecules, 70(4) 391-475 (American Society of Photobiology (1999), the entire disclosure of which is incorporated herein by reference.

Examples of photosensitizers having peak absorptions in visible light and their absorption characteristics is:

| | | | Singlet Oxygen |
|---|---|---|---|
| PS | Lambda Max | Epsilon | QY |
| Methylene Blue | 665 | 48,000 | 0.52 |
| New MB | 630 | 64,000 | N/A |
| Chlorin e6 | 400/650 | 150,000/30,000 | 0.65 |
| Rose Bengal | 562 | 90,000 | 0.76 |
| Protoporphyrin | 409 | 160,000 | 0.91 |
| IX | | | |
| NPe6 | 400/654 | 180,000/40,000 | 0.77 |
| Riboflavin | 460 | 33,000 | 0.54 |
| Curcumin | 430 | 55,000 | N/A |
| Verteporfin | 435 | ~70,000 | N/A |
| Erythrosin B | 530 | 82,000 | 0.63 |
| Eosin Y | 525 | 112,000 | N/A |

Epsilons and QYs for each PS is in their ideal solvent (except for MB, NMB, and Ce6 epsilons we determined in water)

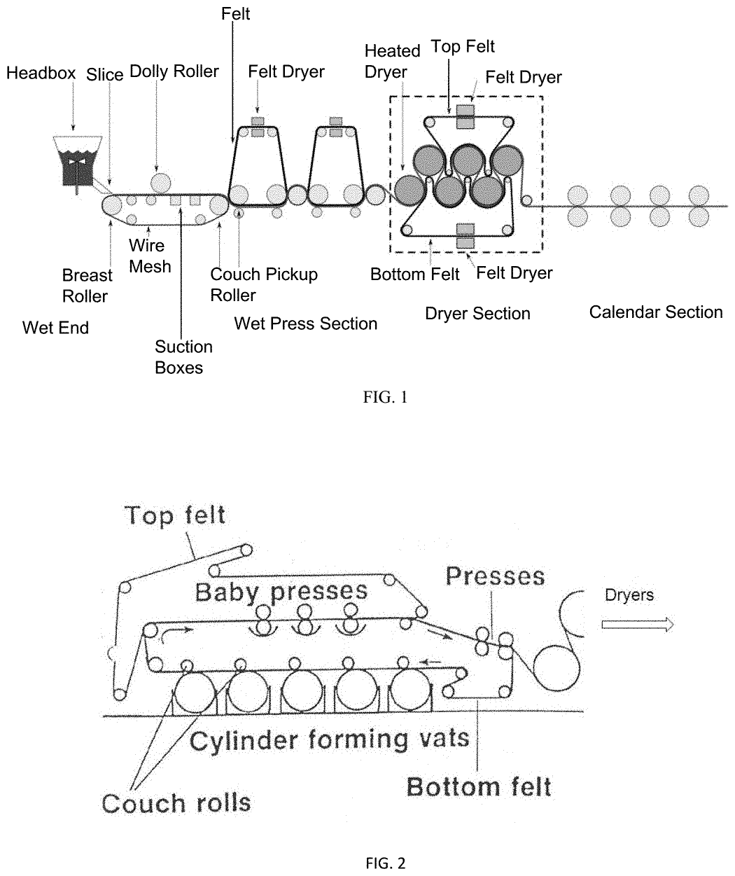

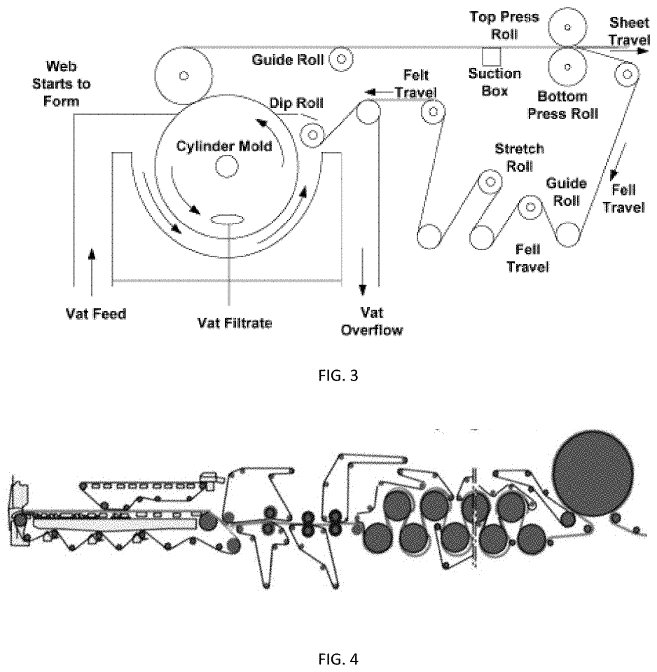

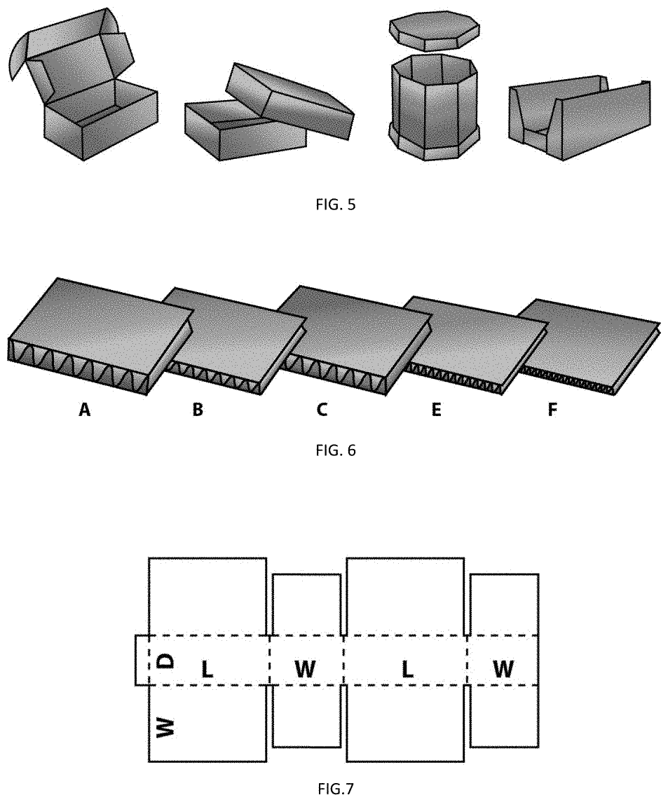

Examples of both non-targeted and targeted nanocompositions (NP-PS) are shown in FIG. 1.

In an embodiment the NP-PS may also be targeted for a specific type of pathogen. The NP-PS may also have a charge, to either assist in the NP-PS linking to a material, e.g., fabric, PPE, non-woven, woven, to provide a targeting or attraction function for a pathogen, and combinations and variations of these.

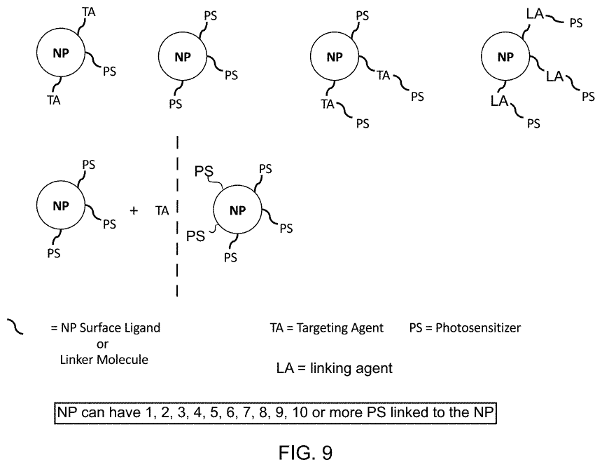

An embodiment of the NP-PS is a targeted delivery of a PS may take several different forms: conjugation of a PS to a nanoparticle (NP), conjugation of a PS to a targeting agent (TA), conjugation of both a PS and TA to a NP (the PS being on the NP, the TA, or both), co-administration of a PS (with or without a NP) with a TA, or any combination thereof. Examples of some of these configurations for the present nanocompositions is shown in FIG. 9.

PSTAs include, for example, a small molecule, a protein, a peptide, an enzyme substrate, a hormone, an antibody, an antigen, a hapten, an avidin, a streptavidin, biotin, a carbohydrate, an oligosaccharide, a polysaccharide, a nucleic acid, a deoxy nucleic acid, a fragment of DNA, a fragment of RNA, nucleotide triphosphates, acyclo terminator triphosphates, peptide nucleic acid (PNA) biomolecules, and combinations and variations of these.

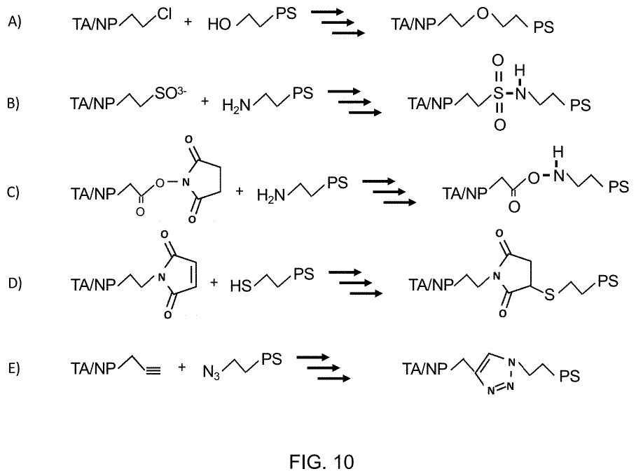

Turning to FIG. 10 there is shown embodiments of methods by which a PS may be covalently conjugated to a TA or an NP. These methods are useful and applicable across most combinations, and so they are generally discussed as if they are a single method. Thus, any given method of NP conjugation should also be viable for TA conjugation. It further being understood that as a general requirement the functional groups employed should match each other. Tables 2-4 show a list of pairings and the resulting bonds formed between a TA, NP, or PS for examples of embodiments of combinations for embodiments of the present nanocompositions.

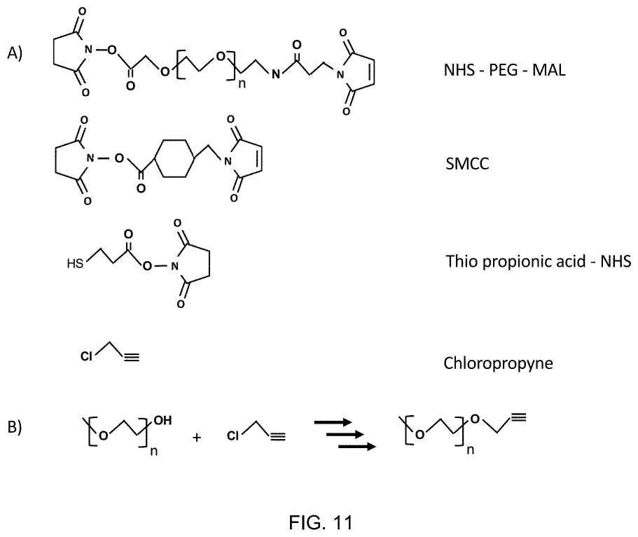

Optionally, conjugation of the PS to a TA, NP, or both, may include a spacer or linker molecule or group. Typically, this will not change the chemistry employed, but it can be used to convert functional groups from one set to another (e.g., an alcohol may be converted to an alkyne with a linking group to enable a different reaction protocol). The linkers may originate on the PS, TA, NP, or any combination, and may be a small molecule chain or polymer. FIG. 11 shows some example linkers and an end group conversion.

An embodiment of a final product would be a NP of small hydrodynamic diameter, preferably from a family of linear, branched, or cyclic macropolymers. Proteins, may also be used as they can be small enough, however, they may have competing pharma co-kinetic behavior with the TA. Examples of macropolymers for the NP would include: polyethylene glycol (PEG), poly amidoamine (PAMAM), polyethyleneimine (PEI), polyvinyl alcohol, and poly L-lysine. The preferred platform is PEG, specifically 8-arm branched PEG (8PEG), because of its widely known non-toxicity. Other nanoparticles may include PVAs (polyvinyl alcohols) and PLGAs (poly(lactic-co-glycolic acid).

The various embodiments of the nanocompositions disclosed and taught herein can use or have multi-arm PEG NPs, this would include 8PEG and other numbers of arms, including 4-arm PEG, including 4PEGA (amine terminated end groups on the arms (one, two and preferably all arms)) and 4PEGMAL (having maleimide terminated end groups on the arms (one, two and preferably all arms)) and 6-arm PEG (including 6PEGA (amine terminated end groups on the arms (one, two and preferably all arms)) and 6PEGMAL (having maleimide terminated end groups on the arms (one, two and preferably all arms)).

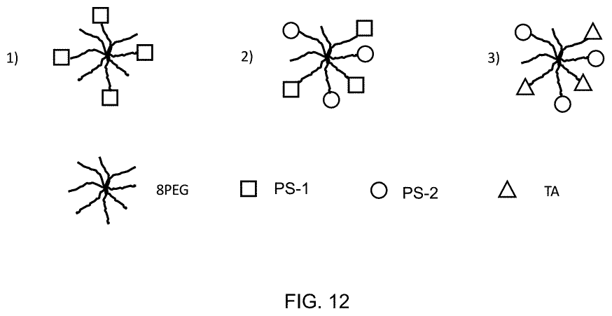

In an embodiment PEG, in particular 8PEG, conjugation can include both a TA and one or more PS, for example, the 3 Forms as shown in FIG. 12.

FIG. 12, Form 1) has a PS (PS-1) that is attached to 8PEGA to provide a TA-PS-NP nanocomposition, having four PS attached to the 8PEGA.

FIG. 12, Form 2) is a PS-1-NP-PS-2 nanocomposition. Form 2) has three PS-1 attached to the 8PEGA, and has three PS-2 attached to the 8PEGA.

FIG. 12, Form 3) is a TA-NP-PA nanocomposition. Form 3) has three PS attached to the 8PEGA, and has three TAs attached to the 8PEGA.

These forms do not have TAs and PSs bonded to every arm of the 8PEGA. Thus, Form 1) has three unbonded, or open, or non-active arms. Forms 2) and 3) have two unbonded, or open, or non-active arms. The unbonded arms, typically have end or terminus groups that are, for example, cysteine.

Additionally, the order of conjugation of the embodiments in FIG. 12 is generally interchangeable.

It is theorized that in uses as a part of a spray on application, or as an additive to a woven or none woven material, as well as other applications, to provide an active surface, active surface layer, active porosity (internal pore surfaces or internal structures), active material or active coatings of the NP-PS nanocomposition to a material the NP serves to space apart and maintain the PS in a configuration that permits the PS to function as an active material to generate RS when exposed to light. Further, this physical spacing (e.g., nano-sized steric considerations) obtained by the NP, in the NP-NS composition, provides for extended periods of time when the PS is active, and increased efficiency in the PS ability to produce ROS.

Additionally, it is theorized that having unbonded or open arms (or areas of the NP) provide the ability for the PS to better, more efficiently (including ROS production and duration of ROS production) function when on the surface. In this manner the individual PS are held apart for each other. It has been discovered that prior attempts of using a PS on a surface failed to provide adequate ROS generation, and failed to provide an acceptable active anti-pathogenic material, because the PS agglomerated, or otherwise could not be maintained in a stable, efficacious or use full configuration when applied to the surface or material. The use of a nanoparticle to space apart or spread the PS from each other overcomes this serious impediment of prior attempts to use PS on surfaces. While having less than all arms of the NP having PS is preferred and optimal, having all arms of the NP having PS will also overcome the problem, and provide the benefits of the present inventions.

Further, the unbonded arms themselves, or they may be functionalized, to provide greater attachment to the surface of the article being treated.

The liquid, e.g., carrier, solvent, also provides the ability to both evenly disperse or deliver the active agent (e.g., the NP-PS, PS or both) to the surface and may prevent or reduce any agglomeration of PS once applied. Preferrably, upon application of the liquid composition to an article, e.g., a surface, agglomeration of the PS is reduced, kept to a minimum and completely avoided.

Contrary to the general teaching of the art, it has been discovered that increasing the number of PS attached to the NP does not necessarily increase the amount of ROS produced, and does not necessarily increase the efficacy of the nanocomposition. Thus, for situations having four or more PS attached to an NP, and in particular 8PEGA, the ROS production and the efficacy of the nanocomposition may be decreased when compared to a nanocomposition having three or less PS. It is theorized that this occurs because of several facts relating to the spacing of the PS, and thus their ability to produce ROS from the in situ oxygen.

Thus, embodiments of NP-PS nanocompositions for PPR have from 1, 2, 3 and 4 PS per 8PEGA. These and other embodiments can have a ratio of open arms (or area) to PS that is 2.5 to 1 and greater, 3 to 1 and greater, and 5 to 1 and greater. These and other embodiments can have 1, 2, 3, and 4 free arms and more. All combinations and variations of these configurations are also contemplated. In other embodiments all arms, (or all available surface area) of the NP has a linked PS.

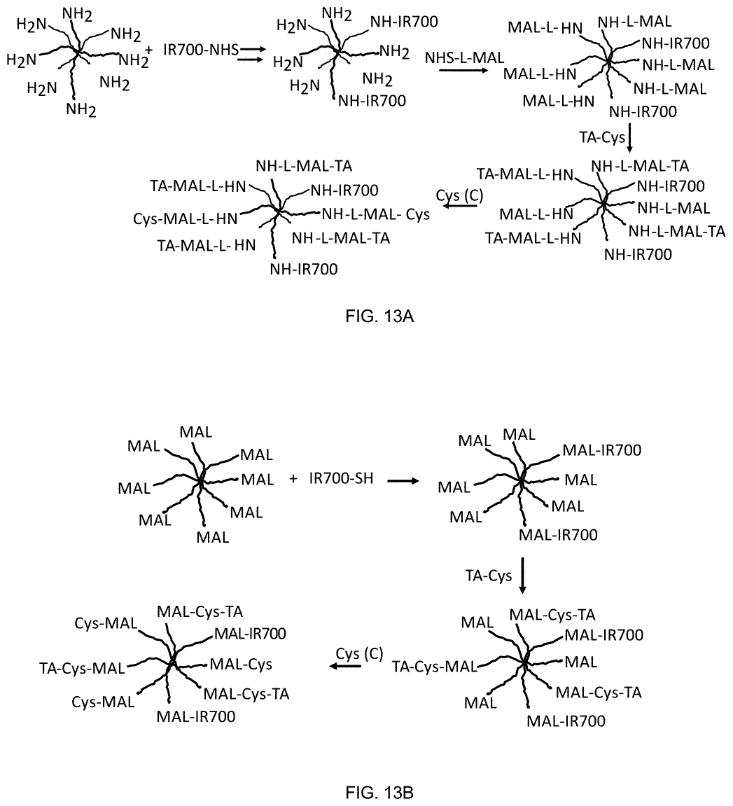

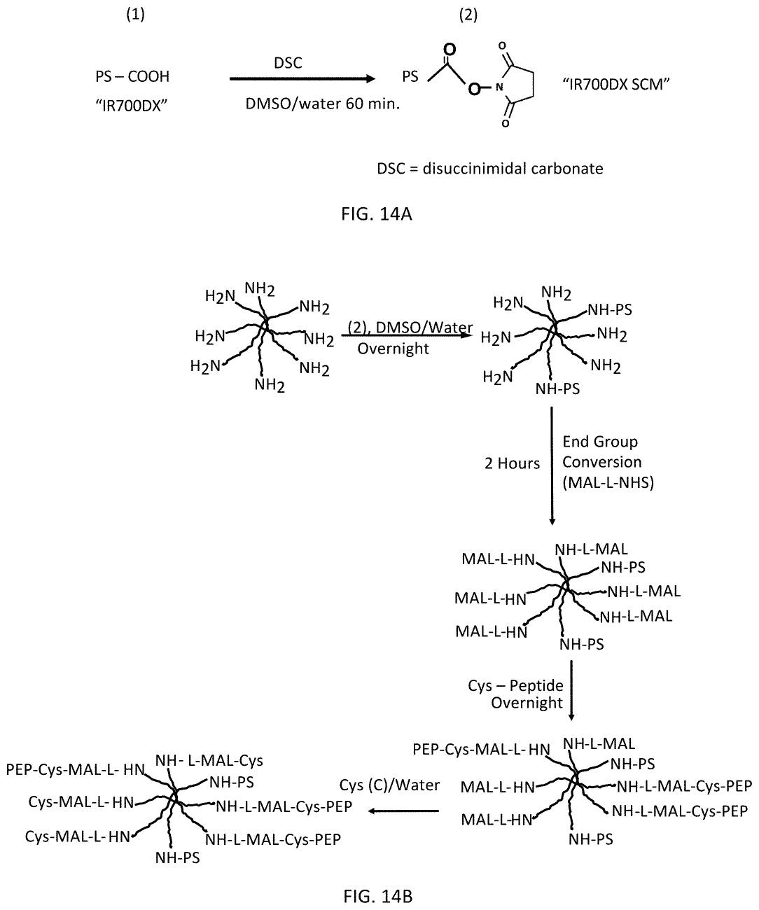

Turning to FIG. 13A there is provided an embodiment of a method to produce the nanocomposition.

FIG. 13A has the following steps:
IR700-NHS is added to 8PEG-Amine (8PEGA)
A linker (L) is added to 8PEGA to convert the amines to maleimides (MAL)
IR700-8PEGM is treated with thiol terminated (preferably cysteine, cys) TA
Additional free cysteine is added to cap unreacted MAL groups

Turning to FIG. 13B there is provided an embodiment of a method to produce the nanocomposition.

FIG. 13B has the following steps:
IR700-SH is added to 8PEGMAL
IR700-8PEGMAL is treated with thiol terminated TA (preferably cysteine, cys)
Additional free cysteine is added to cap unreacted MAL groups

Turning to FIG. 14A and FIG. 14B there is shown a general process for forming targeted nanocompositions for PPR, including an IR700-NP-PTP nanocomposition. “PEP”, (a peptide), is the TA. The end group conversions step of FIG. 14B uses a chemical such as SMCC, BiPEG, or others, that converts the 8PEGA amines to maleimides (“MAL”).

FIG. 14A shows the preparation of the NHS ester (SCM, i.e., succinimidyl ester) for the PS, IR700 (formula (2)). FIG. 14B shows the preparation of the nanocomposition using the HHS ester (FIG. 14A, formula (2)) and a PEP TA.

Covalent conjugation of a NP-X, PS-L-Q, or TA-Z in any combination may take many forms; generally the entities should have X, Q, and Z functional groups that are reactive towards each other. X, Q, and Z include, but are not limited to: alkyl halides, acyl halides, aromatic phenyls, aromatic halides (preferably iodo), carboxylic acids, sulfonic acids, phosphoric acids, alcohols (preferably primary), maleimides, esters, thiols, azides, aldehydes, alkenes (mono or diene), isocyanates, isothiocyanates, amines, anhydrides, or thiols. Tables 2-4 show the matching relevant combinations of NP-X, PS-L-Q, and TA-Z functional groups for conjugation.

TABLE 2
X and Q pairings of NP-X and PS-L-Q for
covalent conjugation (Makes PS(L)-NP-X)

| NP-X | PS-L-Q | Conditions | Covalent Bond |
|---|---|---|---|
| Alkyl Halide | PS-OH | Base, CHCl₃ or | Ether |
| (Chlorine) | PS-SH | DMSO | Thio Ether |
| | PS-COOH | | Ester |
| | PS-NH₂ | | |
| Acyl Halide | PS-NH₂ | 1.5:1 Base:PS-Y | Amide |
| (Chlorine) | PS-SH | (Opt) CHCl₃ | Thio Ester |
| | PS-OH | or DMSO | Ester |
| | PS-Phenyl | | Ketone |
| Aromatic | PS-Cl | AlCl₃, CHCl₃ or | Alkyl chain |
| (Phenyl) | PS-COCl | DMSO | ketone |
| Aromatic | PS-NH₂ | Base, CHCl₃ or | Secondary Amine |
| (Halide | PS-OH | DMSO | Ether |
| Phenyl) | PS-SH | | Thioether |
| Carboxylic | PS-OH | Acid, CHCl₃ or | Ester |
| Acid | PS-NH₂ | DMSO; | Amide |
| | PS-Cl | Acid, CHCl₃ or | Ester |
| | PS-SH | DMSO; | Thioester |
| | | Base, CHCl₃ or | |
| | | DMSO; | |
| | | Acid, CHCl₃ or | |
| | | DMSO | |
| Sulfonic | PS-OH | 1.5:1 Base:PS-Y | Sulfonic ester |
| Acid | PS-NH₂ | PCl₅, CHCl₃ or | Amino Sulfonate |
| | PS-SH | DMSO; | Sulfonic thioester |
| | | SOCl₂ may also be | |
| | | used | |
| Phosphoric | PS-OH | 1.5:1 Base:PS-Y | Phosphoramidite |
| Acid | PS-NH₂ | SOCl₂, CHCl₃ or | |
| | PS-SH | DMSO | |
| Alcohol | PS-Cl | Base, CHCl₃ or | Ether |
| (Primary) | PS-COOH | DMSO; | Ester |
| | PS-ester | Base, CHCl₃ or | Ester |
| | PS-thioester | DMSO; | Ester |
| | PS-anhydride | Base, CHCl₃ or | Ester |
| | PS-CHO | DMSO; | Ester |
| | PS-ITC | Base, CHCl₃ or | Thiocarbamate |
| | PS-IC | DMSO; | Urethane |
| | | Base, CHCl₃ or | |
| | | DMSO; | |
| | | Base, Pd catalyst, | |
| | | CHCl₃; | |
| | | 1.5:1 Base:PS-Y, | |
| | | CHCl₃; | |
| | | 1.5:1 Base:PS-Y, | |
| | | CHCl₃ | |
| Maleimide | PS-SH | pH 6-8 in water; | Thioether |
| (MAL) | | 1.5:1 Base:PS-Y in | |
| | | organic solvent | |
| Ester | PS-NH₂ | Acid, CHCl₃ or | Amide |
| | PS-OH | DMSO | Ester |
| | PS-SH | | Thioester |
| Thiol | PS-Mal | pH 6-8 in water; | Thioether |
| | PS-ITC | 1.5:1 Base:PS-Y, | Dithiocarbamate |
| | PS-IC | CHCl₃; | Thiourethane |
| | | 1.5:1 Base:PS-Y, | |
| | | CHCl₃ | |
| Azide | PS-Alkyne | Cu(I), CHCl₃ or | Triazole |
| | | DMSO; | |
| | | Cu free, CHCl₃ or | |
| | | water | |
| Aldehyde | PS-NH2 | CuI, TBHP, CHCl3; | Amide |
| | PS-OH | Base, Pd catalyst, | Ester |
| | | CHCl₃; | |
| Alkene | PS-Diene | Diels-Alder | Cyclo-alkyl |
| Alkyne | PS-Azide | Cu(I), CHCl₃ or | Triazole |
| | | DMSO; | |
| | | Cu free, CHCl₃ or | |
| | | water | |
| isocyanate | PS-OH | Base, CHCl₃; | Urethane |
| | PS-NH₂ | CHCl₃; | Urea |
| | PS-SH | Base, CHCl₃ | Thiourethane |
| isothiocyanate | PS-SH | 1.5:1 Base:PS-Y, | Dithiocarbamate |
| | PS-NH₂ | CHCl₃; | Thiourea |
| | PS-OH | pH 7.4 in water; | Thiocarbamate |
| | | 1.5:1 Base:PS-Y, | |
| | | CHCl₃ | |
| Amine (A) | PS-COOH | Acid, CHCl₃ or | Amide |
| | PS-COCl | DMSO; | Amide |
| | PS-NHS | Base (Opt), CHCl₃ | Amide |
| | PS-CHO | pH 7.4 in water; | Amide |
| | PS-ITC | Base, Pd catalyst, | Thiourea |
| | PS-IC | CHCl₃; | Urea |
| | | pH 7.4 in water; | |
| | | pH 7.4 in water | |
| Anhydride | PS-NH₂ | CHCl3 or DMSO; | Amide |
| | PS-OH | 1.5:1 Base:PS-Y, | Ester |
| | PS-SH | CHCl₃; | Thioester |
| | | 1.5:1 Base:PS-Y, | |
| | | CHCl₃ | |
| Thiol | PS-SH | Oxidant, CHCl₃ | Disulfide |

*Opt = optional;
NHS = N-hydroxy succinimide;
ITC = isothiocycanate;
IC = isocyanate

TABLE 3
X and Z pairings of PS(L)-NP-X or NP-X alone and
TA-Z for covalent conjugation (to make PS(L)-
NP-TA the preferred material or NP-TA alone)

| PS(L)-NP-X | | | |
|---|---|---|---|
| (or NP-X) | TA-Z | Conditions | Covalent Bond |
| Alkyl Halide | TA-OH | Base, CHCl₃ or | Ether |
| (Chlorine) | TA-SH | DMSO | Thio Ether |
| | TA-COOH | | Ester |
| | TA-NH₂ | | |
| Acyl Halide | TA-NH₂ | 1.5:1 Base:PS-Y | Amide |
| (Chlorine) | TA-SH | (Opt) CHCl₃ | Thio Ester |
| | TA-OH | or DMSO | Ester |
| | TA-Phenyl | | Ketone |
| Aromatic | TA-Cl | AlCl₃, | Alkyl chain |
| (Phenyl) | TA-COCl | CHCl₃ or | ketone |
| | | DMSO | |
| Aromatic | TA-NH₂ | Base, CHCl₃ | Secondary Amine |
| (Halide | TA-OH | or DMSO | Ether |
| Phenyl) | TA-SH | | Thioether |
| Carboxylic | TA-OH | Acid, CHCl₃ | Ester |
| Acid | TA-NH₂ | or DMSO; | Amide |
| | TA-Cl | Acid, CHCl₃ | Ester |
| | TA-SH | or DMSO; | Thioester |
| | | Base, CHCl₃ | |
| | | or DMSO; | |
| | | Acid, CHCl₃ | |
| | | or DMSO | |
| Sulfonic | TA-OH | 1.5:1 Base:PS-Y | Sulfonic ester |
| Acid | TA-NH₂ | PCl₅, | Amino Sulfonate |
| | TA-SH | CHCl₃ | Sulfonic thioester |
| | | or DMSO; | |
| | | SOCl₂ may | |
| | | also be used | |
| Phosphoric | TA-OH | 1.5:1 Base:PS-Y | Phosphoramidite |
| Acid | TA-NH₂ | SOCl₂, | |
| | TA-SH | CHCl₃ | |
| | | or DMSO | |
| Alcohol | TA-Cl | Base, CHCl₃ | Ether |
| (Primary) | TA-COOH | or DMSO; | Ester |
| | TA-ester | Base, CHCl₃ | Ester |
| | TA-thioester | or DMSO; | Ester |
| | TA-anhydride | Base, CHCl₃ | Ester |
| | TA-CHO | or DMSO; | Ester |
| | TA-ITC | Base, CHCl₃ | Thiocarbamate |
| | TA-IC | or DMSO; | Urethane |
| | | Base, CHCl₃ | |
| | | or DMSO; | |
| | | Base, Pd catalyst, | |
| | | CHCl₃; | |
| | | 1.5:1 Base:PS-Y, | |
| | | CHCl₃; | |
| | | 1.5:1 Base:PS-Y, | |
| | | CHCl₃ | |
| Maleimide | TA-SH | pH 6-8 in water; | Thioether |
| (Mal) | | 1.5:1 Base:PS-Y in | |
| | | organic solvent | |
| Ester | TA-NH₂ | Acid, CHCl₃ | Amide |
| | TA-OH | or DMSO | Ester |
| | TA-SH | | Thioester |
| Thiol | TA-Mal | pH 6-8 in water; | Thioether |
| | TA-ITC | 1.5:1 Base:PS-Y, | Dithiocarbamate |
| | TA-IC | CHCl₃; | Thiourethane |
| | | 1.5:1 Base:PS-Y, | |
| | | CHCl₃ | |
| Azide | TA-Alkyne | Cu(I), CHCl₃ | Triazole |
| | | or DMSO; | |
| | | Cu free, CHCl₃ | |
| | | or water | |
| Aldehyde | TA-NH2 | CuI, TBHP, CHCl3; | Amide |
| | TA-OH | Base, Pd catalyst, | Ester |
| | | CHCl₃; | |
| Alkene | TA-Diene | Diels-Alder | Cyclo-alkyl |
| Alkyne | TA-Azide | Cu(I), CHCl₃ | Triazole |
| | | or DMSO; | |
| | | Cu free, CHCl₃ | |
| | | or water | |
| isocyanate | TA-OH | Base, CHCl₃; | Urethane |
| | TA-NH₂ | CHCl₃; | Urea |
| | TA-SH | Base, CHCl₃ | Thiourethane |
| isothiocyanate | TA-SH | 1.5:1 Base:PS-Y, | Dithiocarbamate |
| | TA-NH₂ | CHCl₃; | Thiourea |
| | TA-OH | pH 7.4 in water; | Thiocarbamate |
| | | 1.5:1 Base:PS-Y, | |
| | | CHCl₃ | |
| Amine (A) | TA-COOH | Acid, CHCl₃ | Amide |
| | TA-COCl | or DMSO; | Amide |
| | TA-NHS | Base (Opt), CHCl₃ | Amide |
| | TA-CHO | pH 7.4 in water; | Amide |
| | TA-ITC | Base, Pd catalyst, | Thiourea |
| | TA-IC | CHCl₃; | Urea |
| | | pH 7.4 in water; | |
| | | pH 7.4 in water | |
| Anhydride | TA-NH₂ | CHCl3 or DMSO; | Amide |
| | TA-OH | 1.5:1 Base:PS-Y, | Ester |
| | TA-SH | CHCl₃; | Thioester |
| | | 1.5:1 Base:PS-Y, | |
| | | CHCl₃ | |
| Thiol | TA-SH | Oxidant, CHCl₃ | Disulfide |

*Opt = optional;
NHS = N-hydroxy succinimide;
ITC = isothiocycanate;
IC = isocyanate

TABLE 4
Q and Z pairings of PS-L-Q and TA-Z for covalent conjugation
(This makes PS(L)-TA, that could potentially be used
(no NP) or could then be attached to the NP to form
a new (and never tried) form PA-TS-NP)

| PS-L-Q | TA-Z | Conditions | Covalent Bond |
|---|---|---|---|
| Alkyl Halide | TA-OH | Base, CHCl₃ or | Ether |
| (Chlorine) | TA-SH | DMSO | Thio Ether |
| | TA-COOH | | Ester |
| | TA-NH₂ | | |
| Acyl Halide | TA-NH₂ | 1.5:1 Base:PS-Y | Amide |
| (Chlorine) | TA-SH | (Opt) CHCl₃ | Thio Ester |
| | TA-OH | or DMSO | Ester |
| | TA-Phenyl | | Ketone |
| Aromatic | TA-Cl | AlCl₃, CHCl₃ or | Alkyl chain |
| (Phenyl) | TA-COCl | DMSO | ketone |
| Aromatic | TA-NH₂ | Base, CHCl₃ or | Secondary Amine |
| (Halide | TA-OH | DMSO | Ether |
| Phenyl) | TA-SH | | Thioether |
| Carboxylic | TA-OH | Acid, CHCl₃ or | Ester |
| Acid | TA-NH₂ | DMSO; | Amide |
| | TA-Cl | Acid, CHCl₃ or | Ester |
| | TA-SH | DMSO; | Thioester |
| | | Base, CHCl₃ or | |
| | | DMSO; | |
| | | Acid, CHCl₃ or | |
| | | DMSO | |
| Sulfonic | TA-OH | 1.5:1 Base:PS-Y | Sulfonic ester |
| Acid | TA-NH₂ | PCl₅, CHCl₃ or | Amino Sulfonate |
| | TA-SH | DMSO; | Sulfonic thioester |
| | | SOCl₂ may also be | |
| | | used | |
| Phosphoric | TA-OH | 1.5:1 Base:PS-Y | Phosphoramidite |
| Acid | TA-NH₂ | SOCl₂, CHCl₃ or | |
| | TA-SH | DMSO | |
| Alcohol | TA-Cl | Base, CHCl₃ or | Ether |
| (Primary) | TA-COOH | DMSO; | Ester |
| | TA-ester | Base, CHCl₃ or | Ester |
| | TA-thioester | DMSO; | Ester |
| | TA-anhydride | Base, CHCl₃ or | Ester |
| | TA-CHO | DMSO; | Ester |
| | TA-ITC | Base, CHCl₃ or | Thiocarbamate |
| | TA-IC | DMSO; | Urethane |
| | | Base, CHCl₃ or | |
| | | DMSO; | |
| | | Base, Pd catalyst, | |
| | | CHCl₃; | |
| | | 1.5:1 Base:PS-Y, | |
| | | CHCl₃; | |
| | | 1.5:1 Base:PS-Y, | |
| | | CHCl₃ | |
| Maleimide | TA-SH | pH 6-8 in water; | Thioether |
| (Mal) | | 1.5:1 Base:PS-Y in | |
| | | organic solvent | |
| Ester | TA-NH₂ | Acid, CHCl₃ or | Amide |
| | TA-OH | DMSO | Ester |
| | TA-SH | | Thioester |
| Thiol | TA-Mal | pH 6-8 in water; | Thioether |
| | TA-ITC | 1.5:1 Base:PS-Y, | Dithiocarbamate |
| | TA-IC | CHCl₃; | Thiourethane |
| | | 1.5:1 Base:PS-Y, | |
| | | CHCl₃ | |
| Azide | TA-Alkyne | Cu(I), CHCl₃ or | Triazole |
| | | DMSO; | |
| | | Cu free, CHCl₃ or | |
| | | water | |
| Aldehyde | TA-NH2 | CuI, TBHP, CHCl3; | Amide |
| | TA-OH | Base, Pd catalyst, | Ester |
| | | CHCl₃; | |
| Alkene | TA-Diene | Diels-Alder | Cyclo-alkyl |
| Alkyne | TA-Azide | Cu(I), CHCl₃ or | Triazole |
| | | DMSO; | |
| | | Cu free, CHCl₃ or | |
| | | water | |
| isocyanate | TA-OH | Base, CHCl₃; | Urethane |
| | TA-NH₂ | CHCl₃; | Urea |
| | TA-SH | Base, CHCl₃ | Thiourethane |
| isothiocyanate | TA-SH | 1.5:1 Base:PS-Y, | Dithiocarbamate |
| | TA-NH₂ | CHCl₃; | Thiourea |
| | TA-OH | pH 7.4 in water; | Thiocarbamate |
| | | 1.5:1 Base:PS-Y, | |
| | | CHCl₃ | |
| Amine (A) | TA-COOH | Acid, CHCl₃ or | Amide |
| | TA-COCl | DMSO; | Amide |
| | TA-NHS | Base (Opt), CHCl₃ | Amide |
| | TA-CHO | pH 7.4 in water; | Amide |
| | TA-ITC | Base, Pd catalyst, | Thiourea |
| | TA-IC | CHCl₃; | Urea |
| | | pH 7.4 in water; | |
| | | pH 7.4 in water | |
| Anhydride | TA-NH₂ | CHCl3 or DMSO; | Amide |
| | TA-OH | 1.5:1 Base:PS-Y, | Ester |
| | TA-SH | CHCl₃; | Thioester |
| | | 1.5:1 Base:PS-Y, | |
| | | CHCl₃ | |
| Thiol | TA-SH | Oxidant, CHCl₃ | Disulfide |

*Opt = optional;
NHS = N-hydroxy succinimide;
ITC = isothiocycanate;
IC = isocyanate

In general, the photosensitizer, the nanoparticle photosensitizer composite, and both, can be added to a liquid and then the liquid can be applied to the article to be treated. One or more different types of photosensitizers and nanoparticle photosensitizer compositions can be added to a liquid. Generally, the liquid, some to all, will evaporate leaving the nanoparticle photosensitizer on the article providing an active antipathogenic surface upon exposure to an activation illumination. Preferably, the surface of the article has about 80% of its surface area covered with the liquid, 90% of the surface covered with the liquid, and 100% of the surface covered with the liquid. The surface of the article has about 25% to about 100%, about 25% or more, about 50% or more, about 70% or more, and about 90% or more of its surface covered with the nanoparticle photosensitizer.

In general, the photosensitizer, the nanoparticle photosensitizer composite, and both, can be added to a liquid and then the liquid can be freeze dried or concentrated, for later use, or making down into a liquid for use, e.g., spraying on articles.

The liquid can be a mixture of from 0 to 100% of water, 0 to 100% cyclodextrin and 0 to 100% alcohol and 0 to 50% of other materials.

In embodiments, the liquid which can be a carrier, solvent, or both, for the photosensitizer, nanoparticle photosensitizer composite, to deliver the active components. In embodiments their can 5% or less alcohol to 70% of more. Preferably, the liquid, e.g., solvent system, is chosen to ensure the product is stable in storage and use and that once used provides its purpose as a carrier and then simply and safely evaporates.

It being understood that while IR-700 is used as an example, or used in several of the Examples of this Specification, the methods and techniques used for forming these NP-PS nanocomposites are general methods, application to other PSs, and other NPs. These methods and techniques can be used to form, and are applicable to form, any NP-PS nanocomposite using any of the NPs and PSs disclosed and taught by this specification.

In an embodiment the composition changes color as the PS is used up, providing a visual indication that a second treatment (re-treatment) with the PS composition is required. The visual indicator can be from the PS itself, or can be from a separate dye that changes color upon the reduction or sensation of ROS production, i.e., the PS is no longer active.

In an embodiment, the liquid, the PS composition, and combinations and variations of these are free from any material that would quench the PS, or otherwise interfere with the production or ROS.

In an embodiment the PS-composition is a concentrate, e.g., high solids liquid concentrate, lyophilized concentrate, which is then diluted prior to or upon use. The concentrate can be contained in sackets, or pods, of water-soluble film. The pods are then placed in water, and the dilute solution applied, e.g., rolled, wiped, sprayed, to an article, e.g., PPE.

Generally, in the PS-ICF formulations the PS is associated with the ICF. Typically, this association is by way of Van der Waals forces. In embodiments this association may be steric (e.g., steric hinderance), non-covalent, covalent and other forms of linking the PS with or to the ICF. In these formulations the ICF can also be associated with NP, NP-TA compositions. Preferably the ICF-NP, ICF-NP-TA association is by way of a covalent bond. In embodiments other types of association may also be used.

In embodiments the ICF can have two, three or more PS associated with it. The PS in this multi-PS ICF complex can be the same PS or they can be different.

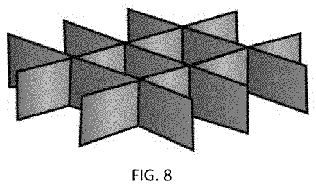

Turning to FIG. 8 there is shown embodiments of methods by which a PS may be associated with a covalently conjugated to a ICF-NP composition. These methods are useful and applicable across most combinations, and so they are generally discussed as if they are a single method. Thus, any given method of NP conjugation should also be viable for ICF conjugation. It further being understood that as a general requirement the functional groups employed should match each other. Tables 2A, 3A and 4A show a list of pairings and the resulting bonds formed between a TA, NP, or ICF for examples of embodiments of combinations for embodiments of the present ICF-PS, and ICF-PS nanocompositions.

For example, a covalent conjugation for ICFs can be a NP-X, ICF-L-Q, or TA-Z in any combination and may take many forms; generally the entities should have X, Q, and Z functional groups that are reactive towards each other. X, Q, and Z include, but are not limited to: alkyl halides, acyl halides, aromatic phenyls, aromatic halides (preferably iodo), carboxylic acids, sulfonic acids, phosphoric acids, alcohols (preferably primary), maleimides, esters, thiols, azides, aldehydes, alkenes (mono or diene), isocyanates, isothiocyanates, amines, anhydrides, or thiols. Tables 2A, 3A and 4A show the matching relevant combinations of NP-X, ICF-L-Q, and TA-Z functional groups for conjugation.

TABLE 2A
X and Q pairings of NP-X and ICF-L-Q for
covalent conjugation (Makes ICF(L)-NP-X)

| NP-X | ICF-L-Q | Conditions | Covalent Bond |
|---|---|---|---|
| Alkyl Halide | ICF-OH | Base, CHCl₃ or | Ether |
| (Chlorine) | ICF-SH | DMSO | Thio Ether |
| | ICF-COOH | | Ester |
| | ICF-NH₂ | | |
| Acyl Halide | ICF-NH₂ | 1.5:1 Base:ICF-Y | Amide |
| (Chlorine) | ICF-SH | (Opt) | Thio Ester |
| | ICF-OH | CHCl₃ or DMSO | Ester |
| | ICF-Phenyl | | Ketone |
| Aromatic | ICF-Cl | AlCl₃, CHCl₃ or | Alkyl chain |
| (Phenyl) | ICF-COCl | DMSO | ketone |
| Aromatic | ICF-NH₂ | Base, CHCl₃ or | Secondary Amine |
| (Halide Phenyl) | ICF-OH | DMSO | Ether |
| | ICF-SH | | Thioether |
| Carboxylic | ICF-OH | Acid, CHCl₃ or | Ester |
| Acid | ICF-NH₂ | DMSO; | Amide |
| | ICF-C1 | Acid, CHCl₃ or | Ester |
| | ICF-SH | DMSO; | Thioester |
| | | Base, CHCl₃ or | |
| | | DMSO; | |
| | | Acid, CHCl₃ or | |
| | | DMSO | |
| Sulfonic | ICF-OH | 1.5:1 Base:ICF-Y | Sulfonic ester |
| Acid | ICF-NH₂ | PCl₅, CHCl₃ or | Amino Sulfonate |
| | ICF-SH | DMSO; | Sulfonic thioester |
| | | SOCl₂ may also be | |
| | | used | |
| Phosphoric | ICF-OH | 1.5:1 Base:ICF-Y | Phosphoramidite |
| Acid | ICF-NH₂ | SOCl₂, CHCl₃ or | |
| | ICF-SH | DMSO | |
| Alcohol | ICF-Cl | Base, CHCl₃ or | Ether |
| (Primary) | ICF-COOH | DMSO; | Ester |
| | ICF-ester | Base, CHCl₃ or | Ester |
| | ICF-thioester | DMSO; | Ester |
| | ICF-anhydride | Base, CHCl₃ or | Ester |
| | ICF-CHO | DMSO; | Ester |
| | ICF-ITC | Base, CHCl₃ or | Thiocarbamate |
| | ICF-IC | DMSO; | Urethane |
| | | Base, CHCl₃ or | |
| | | DMSO; | |
| | | Base, Pd catalyst, | |
| | | CHCl₃; | |
| | | 1.5:1 Base:ICF-Y, | |
| | | CHCl₃; | |
| | | 1.5:1 Base:ICF-Y, | |
| | | CHCl₃ | |
| Maleimide | ICF-SH | pH 6-8 in water; | Thioether |
| (MAL) | | 1.5:1 Base:ICF-Y in | |
| | | organic solvent | |
| Ester | ICF-NH₂ | Acid, CHCl₃ or | Amide |
| | ICF-OH | DMSO | Ester |
| | ICF-SH | | Thioester |
| Thiol | ICF-Mal | pH 6-8 in water; | Thioether |
| | ICF-ITC | 1.5:1 Base:ICF-Y, | Dithiocarbamate |
| | ICF-IC | CHCl₃; | Thiourethane |
| | | 1.5:1 Base:ICF-Y, | |
| | | CHCl₃ | |
| Azide | ICF-Alkyne | Cu(I), CHCl₃ or | Triazole |
| | | DMSO; | |
| | | Cu free, CHCl₃ or | |
| | | water | |
| Aldehyde | ICF-NH2 | CuI, TBHP, CHCl3; | Amide |
| | ICF-OH | Base, Pd catalyst, | Ester |
| | | CHCl₃; | |
| Alkene | ICF-Diene | Diels-Alder | Cyclo-alkyl |
| Alkyne | ICF-Azide | Cu(I), CHCl₃ or | Triazole |
| | | DMSO; | |
| | | Cu free, CHCl₃ or | |
| | | water | |
| isocyanate | ICF-OH | Base, CHCl₃; | Urethane |
| | ICF-NH₂ | CHCl₃; | Urea |
| | ICF-SH | Base, CHCl₃ | Thiourethane |
| isothiocyanate | ICF-SH | 1.5:1 Base:ICF-Y, | Dithiocarbamate |
| | ICF-NH₂ | CHCl₃; | Thiourea |
| | ICF-OH | pH 7.4 in water; | Thiocarbamate |
| | | 1.5:1 Base:ICF-Y, | |
| | | CHCl₃ | |
| Amine (A) | ICF-COOH | Acid, CHCl₃ or | Amide |
| | ICF-COCl | DMSO; | Amide |
| | ICF-NHS | Base (Opt), CHCl₃ | Amide |
| | ICF-CHO | pH 7.4 in water; | Amide |
| | ICF-ITC | Base, Pd catalyst, | Thiourea |
| | ICF-IC | CHCl₃; | Urea |
| | | pH 7.4 in water; | |
| | | pH 7.4 in water | |
| Anhydride | ICF-NH₂ | CHCl3 or DMSO; | Amide |
| | ICF-OH | 1.5:1 Base:ICF-Y, | Ester |
| | ICF-SH | CHCl₃; | Thioester |
| | | 1.5:1 Base:ICF-Y, | |
| | | CHCl₃ | |
| Thiol | ICF-SH | Oxidant, CHCl₃ | Disulfide |

*Opt = optional;
NHS = N-hydroxy succinimide;
ITC = inclusion complex former, e.g., cyclodextrins and derivatives;
IC = isocyanate

TABLE 3A
X and Z pairings of ICF(L)-NP-X or NP-X alone and
TA-Z for covalent conjugation (to make ICF(L)-
NP-TA the preferred material or NP-TA alone)

| PS(L)-NP-X | | | |
|---|---|---|---|
| (or NP-X) | TA-Z | Conditions | Covalent Bond |
| Alkyl Halide | TA-OH | Base, CHCl₃ or | Ether |
| (Chlorine) | TA-SH | DMSO | Thio Ether |
| | TA-COOH | | Ester |
| | TA-NH₂ | | |
| Acyl Halide | TA-NH₂ | 1.5:1 Base:ICF-Y | Amide |
| (Chlorine) | TA-SH | (Opt) | Thio Ester |
| | TA-OH | CHCl₃ or DMSO | Ester |
| | TA-Phenyl | | Ketone |
| Aromatic | TA-Cl | AlCl₃, CHCl₃ or | Alkyl chain |
| (Phenyl) | TA-COCl | DMSO | ketone |
| Aromatic | TA-NH₂ | Base, CHCl₃ or | Secondary Amine |
| (Halide | TA-OH | DMSO | Ether |
| Phenyl) | TA-SH | | Thioether |
| Carboxylic | TA-OH | Acid, CHCl₃ or | Ester |
| Acid | TA-NH₂ | DMSO; | Amide |
| | TA-Cl | Acid, CHCl₃ or | Ester |
| | TA-SH | DMSO; | Thioester |
| | | Base, CHCl₃ or | |
| | | DMSO; | |
| | | Acid, CHCl₃ or | |
| | | DMSO | |
| Sulfonic | TA-OH | 1.5:1 Base:ICF-Y | Sulfonic ester |
| Acid | TA-NH₂ | PCl₅, CHCl₃ or | Amino Sulfonate |
| | TA-SH | DMSO; | Sulfonic thioester |
| | | SOCl₂ may also be | |
| | | used | |
| Phosphoric | TA-OH | 1.5:1 Base:ICF-Y | Phosphoramidite |
| Acid | TA-NH₂ | SOCl₂, CHCl₃ or | |
| | TA-SH | DMSO | |
| Alcohol | TA-Cl | Base, CHCl₃ or | Ether |
| (Primary) | TA-COOH | DMSO; | Ester |
| | TA-ester | Base, CHCl₃ or | Ester |
| | TA-thioester | DMSO; | Ester |
| | TA-anhydride | Base, CHCl₃ or | Ester |
| | TA-CHO | DMSO; | Ester |
| | TA-ITC | Base, CHCl₃ or | Thiocarbamate |
| | TA-IC | DMSO; | Urethane |
| | | Base, CHCl₃ or | |
| | | DMSO; | |
| | | Base, Pd catalyst, | |
| | | CHCl₃; | |
| | | 1.5:1 Base:ICF-Y, | |
| | | CHCl₃; | |
| | | 1.5:1 Base:ICF-Y, | |
| | | CHCl₃ | |
| Maleimide | TA-SH | pH 6-8 in water; | Thioether |
| (Mal) | | 1.5:1 Base:ICF-Y in | |
| | | organic solvent | |
| Ester | TA-NH₂ | Acid, CHCl₃ or | Amide |
| | TA-OH | DMSO | Ester |
| | TA-SH | | Thioester |
| Thiol | TA-Mal | pH 6-8 in water; | Thioether |
| | TA-ITC | 1.5:1 Base:ICF-Y, | Dithiocarbamate |
| | TA-IC | CHCl₃; | Thiourethane |
| | | 1.5:1 Base:ICF-Y, | |
| | | CHCl₃ | |
| Azide | TA-Alkyne | Cu(I), CHCl₃ or | Triazole |
| | | DMSO; | |
| | | Cu free, CHCl₃ or | |
| | | water | |
| Aldehyde | TA-NH2 | CuI, TBHP, CHCl3; | Amide |
| | TA-OH | Base, Pd catalyst, | Ester |
| | | CHCl₃; | |
| Alkene | TA-Diene | Diels-Alder | Cyclo-alkyl |
| Alkyne | TA-Azide | Cu(I), CHCl₃ or | Triazole |
| | | DMSO; | |
| | | Cu free, CHCl₃ or | |
| | | water | |
| isocyanate | TA-OH | Base, CHCl₃; | Urethane |
| | TA-NH₂ | CHCl₃; | Urea |
| | TA-SH | Base, CHCl₃ | Thiourethane |
| isothiocyanate | TA-SH | 1.5:1 Base:ICF-Y, | Dithiocarbamate |
| | TA-NH₂ | CHCl₃; | Thiourea |
| | TA-OH | pH 7.4 in water; | Thiocarbamate |
| | | 1.5:1 Base:ICF-Y, | |
| | | CHCl₃ | |
| Amine (A) | TA-COOH | Acid, CHCl₃ or | Amide |
| | TA-COCl | DMSO; | Amide |
| | TA-NHS | Base (Opt), CHCl₃ | Amide |
| | TA-CHO | pH 7.4 in water; | Amide |
| | TA-ITC | Base, Pd catalyst, | Thiourea |
| | TA-IC | CHCl₃; | Urea |
| | | pH 7.4 in water; | |
| | | pH 7.4 in water | |
| Anhydride | TA-NH₂ | CHCl3 or DMSO; | Amide |
| | TA-OH | 1.5:1 Base:ICF-Y, | Ester |
| | TA-SH | CHCl₃; | Thioester |
| | | 1.5:1 Base:ICF-Y, | |
| | | CHCl₃ | |
| Thiol | TA-SH | Oxidant, CHCl₃ | Disulfide |

*Opt = optional;
NHS = N-hydroxy succinimide;
ITC = inclusion complex former, e.g., cyclodextrins and derivatives;
IC = isocyanate

TABLE 4A
Q and Z pairings of ICF-L-Q and TA-Z for covalent conjugation
(This makes ICF(L)-TA, that could potentially be used
(no NP) or could then be attached to the NP to form
a new (and never tried) form ICF-TS-NP)

| ICF-L-Q | TA-Z | Conditions | Covalent Bond |
|---|---|---|---|
| Alkyl Halide | TA-OH | Base, CHCl₃ or | Ether |
| (Chlorine) | TA-SH | DMSO | Thio Ether |
| | TA-COOH | | Ester |
| | TA-NH₂ | | |
| Acyl Halide | TA-NH₂ | 1.5:1 Base:ICF-Y | Amide |
| (Chlorine) | TA-SH | (Opt) | Thio Ester |
| | TA-OH | CHCl₃ or DMSO | Ester |
| | TA-Phenyl | | Ketone |
| Aromatic | TA-Cl | AlCl₃, CHCl₃ or | Alkyl chain |
| (Phenyl) | TA-COCl | DMSO | ketone |
| Aromatic | TA-NH₂ | Base, CHCl₃ or | Secondary Amine |
| (Halide | TA-OH | DMSO | Ether |
| Phenyl) | TA-SH | | Thioether |
| Carboxylic | TA-OH | Acid, CHCl₃ or | Ester |
| Acid | TA-NH₂ | DMSO; | Amide |
| | TA-Cl | Acid, CHCl₃ or | Ester |
| | TA-SH | DMSO; | Thioester |
| | | Base, CHCl₃ or | |
| | | DMSO; | |
| | | Acid, CHCl₃ or | |
| | | DMSO | |
| Sulfonic | TA-OH | 1.5:1 Base:ICF-Y | Sulfonic ester |
| Acid | TA-NH₂ | PCl₅, CHCl₃ or | Amino Sulfonate |
| | TA-SH | DMSO; | Sulfonic thioester |
| | | SOCl₂ may also be | |
| | | used | |
| Phosphoric | TA-OH | 1.5:1 Base:ICF-Y | Phosphoramidite |
| Acid | TA-NH₂ | SOCl₂, CHCl₃ or | |
| | TA-SH | DMSO | |
| Alcohol | TA-Cl | Base, CHCl₃ or | Ether |
| (Primary) | TA-COOH | DMSO; | Ester |
| | TA-ester | Base, CHCl₃ or | Ester |
| | TA-thioester | DMSO; | Ester |
| | TA-anhydride | Base, CHCl₃ or | Ester |
| | TA-CHO | DMSO; | Ester |
| | TA-ITC | Base, CHCl₃ or | Thiocarbamate |
| | TA-IC | DMSO; | Urethane |
| | | Base, CHCl₃ or | |
| | | DMSO; | |
| | | Base, Pd catalyst, | |
| | | CHCl₃; | |
| | | 1.5:1 Base:ICF-Y, | |
| | | CHCl₃; | |
| | | 1.5:1 Base:ICF-Y, | |
| | | CHCl₃ | |
| Maleimide | TA-SH | pH 6-8 in water; | Thioether |
| (Mal) | | 1.5:1 Base:ICF-Y in | |
| | | organic solvent | |
| Ester | TA-NH₂ | Acid, CHCl₃ or | Amide |
| | TA-OH | DMSO | Ester |
| | TA-SH | | Thioester |
| Thiol | TA-Mal | pH 6-8 in water; | Thioether |
| | TA-ITC | 1.5:1 Base:ICF-Y, | Dithiocarbamate |
| | TA-IC | CHCl₃; | Thiourethane |
| | | 1.5:1 Base:ICF-Y, | |
| | | CHCl₃ | |
| Azide | TA-Alkyne | Cu(I), CHCl₃ or | Triazole |
| | | DMSO; | |
| | | Cu free, CHCl₃ or | |
| | | water | |
| Aldehyde | TA-NH2 | CuI, TBHP, CHCl₃; | Amide |
| | TA-OH | Base, Pd catalyst, | Ester |
| | | CHCl₃; | |
| Alkene | TA-Diene | Diels-Alder | Cyclo-alkyl |
| Alkyne | TA-Azide | Cu(I), CHCl₃ or | Triazole |
| | | DMSO; | |
| | | Cu free, CHCl₃ or | |
| | | water | |
| isocyanate | TA-OH | Base, CHCl₃; | Urethane |
| | TA-NH₂ | CHCl₃; | Urea |
| | TA-SH | Base, CHCl₃ | Thiourethane |
| isothiocyanate | TA-SH | 1.5:1 Base:ICF-Y, | Dithiocarbamate |
| | TA-NH₂ | CHCl₃; | Thiourea |
| | TA-OH | pH 7.4 in water; | Thiocarbamate |
| | | 1.5:1 Base:ICF-Y, | |
| | | CHCl₃ | |
| Amine (A) | TA-COOH | Acid, CHCl₃ or | Amide |
| | TA-COCl | DMSO; | Amide |
| | TA-NHS | Base (Opt), CHCl₃ | Amide |
| | TA-CHO | pH 7.4 in water; | Amide |
| | TA-ITC | Base, Pd catalyst, | Thiourea |
| | TA-IC | CHCl₃; | Urea |
| | | pH 7.4 in water; | |
| | | pH 7.4 in water | |
| Anhydride | TA-NH₂ | CHCl3 or DMSO; | Amide |
| | TA-OH | 1.5:1 Base:ICF-Y, | Ester |
| | TA-SH | CHCl₃; | Thioester |
| | | 1.5:1 Base:ICF-Y, | |
| | | CHCl₃ | |
| Thiol | TA-SH | Oxidant, CHCl₃ | Disulfide |

*Opt = optional;
NHS = N-hydroxy succinimide;
ITC = inclusion complex former, e.g., cyclodextrins and derivatives;
IC = isocyanate

In general, the PS-ICF, can be added to a liquid and then the liquid can be applied to the article to be treated. One or more different types of photosensitizers and nanoparticle photosensitizer compositions can be added to a liquid. Generally, the liquid, some to all, will evaporate leaving the nanoparticle photosensitizer on the article providing an active antipathogenic surface upon exposure to an activation illumination. Preferably, the surface of the article has about 80% of its surface area covered with the liquid, 90% of the surface covered with the liquid, and 100% of the surface covered with the liquid. The surface of the article has about 25% to about 100%, about 25% or more, about 50% or more, about 70% or more, and about 90% or more of its surface covered with the nanoparticle photosensitizer.

In general, the PS-ICF, the nanoparticle photosensitizer composite, and both, can be added to a liquid and then the liquid can be freeze dried or concentrated, for later use, or making down into a liquid for use, e.g., spraying on articles.

The liquid can be a mixture of from 0 to 100% of water, 0 to 100% cyclodextrin and 0 to 100% alcohol and 0 to 50% of other materials.

In embodiments, the liquid which can be a carrier, solvent, or both, for the PS-ICF composite, to deliver the active components. In embodiments their can 5% or less alcohol to 70% of more. Preferably, the liquid, e.g., solvent system, is chosen to ensure the product is stable in storage and use and that once used provides its purpose as a carrier and then simply and safely evaporates.

### Photodynamic Effect.

Generally, the present formulations and compositions use photodynamic effect (or photosensitization) to produce ROS (see Figure A). ROS kills the pathogen, by disrupting lipid capsules, destroy proteins and DNA and RNA structures. Although this specification primarily focuses on Covid-19, the present formulations, methods, compositions, coatings and ROS are effective against almost all bacteria (gram + and gram −), viruses, and other pathogens. These approaches also ensure reduce the ability of the pathogen becoming “resistance” to the active agent, and preferably there is no opportunity for resistance to appear in the pathogen, no formation a resistant pathogen.

Generally, “light”, “illumination” and how they interact with molecules can effect the operation of the present compositions, formulations and methods. Three parameters are generally considered (although others may be evaluated); the wavelength (energy) of the light (nm), the power of the light per unit time (J/s) and the total exposure (dose) per unit area (J/m²).

Light is electromagnetic radiation and generally refers to visible light, extending from approximately 380 nm-740 nm (blue to red). Different wavelengths of light have different energies; blue light is higher energy than red light. Light is also “quantized”, meaning that it delivers defined packets of energy (photons), the value of which decreases as the wavelength increases—these are described by the equations below.

Eₚₕ=hv (1)

v=c/λ (2)

Eₚₕ=hc/λ

Eₚₕ is the energy of a single photon and is measured in Joules (J), h is Planck's constant, λ is the wavelength of the light, v is the frequency of the light and c is the speed of light. This is important in considering how light interacts with a photosensitizer, as “excitation” is also quantized—and thus a photosensitizer can only work if exposed to the correct wavelength of light. This is important in choosing the right photosensitizer for the right environment.

The other key consideration is the power of that light, perhaps more simply stated as how much light energy (per unit time is delivered−J/s or Watts) this is essentially “brightness” and when considering light striking a surface “illuminance”. Illuminance is measured in Lux and sometimes Lumens (1 Lux=1 Lumen/m²). For practical purposes and discussion—the higher the Lux the brighter the light. Most healthcare facilities operate around 1,000 Lux for general care areas and up to 30,000 Lux for operating suites (a bright sunny day can be up to 100,000 Lux in the sun and 20,000 Lux in the shade).

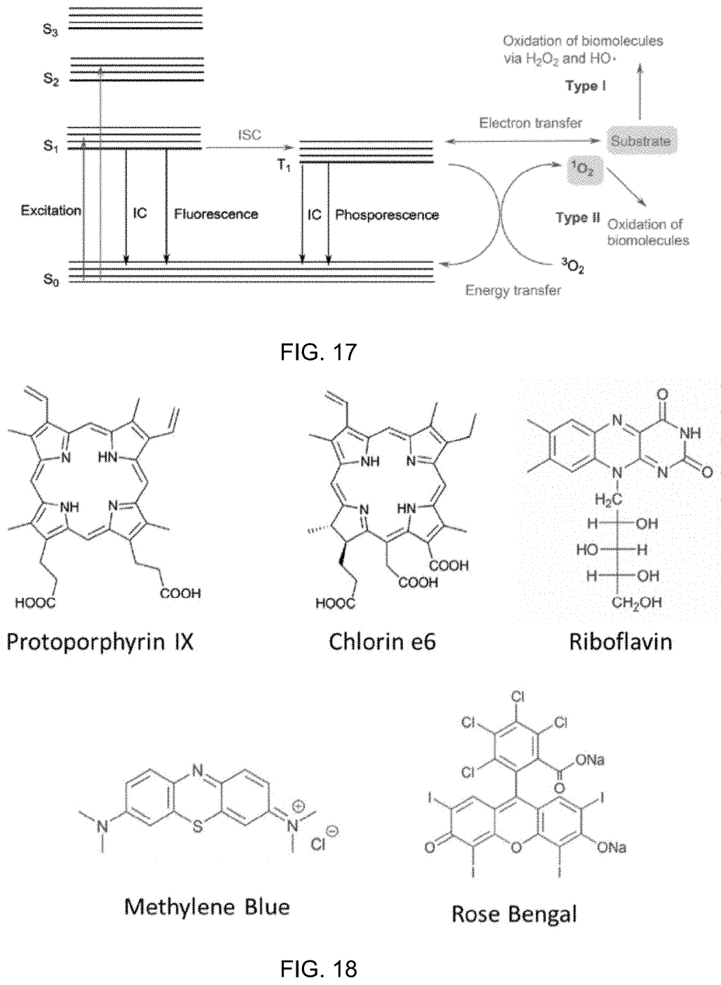

Taking this and applying this to the photodynamic effect, there is determined the photosensitizer the “right” wavelength of light and enough of that light for a sufficient period of time to drive the effect that we want to see, this process is best described for a photosensitizer in terms of electronic transitions (referred to as a Modified Jablonski Diagram and shown in FIG. 17 as a schematic representation of the production of ROS.

Typically, the photodynamic process proceeds via the following steps:

Absorption of a specific wavelength of light (quantized) that excites an electron from the S₀ to S₁ or higher excited state—these are referred to a singlet states and have short (nanosecond) lifetimes before they either decay either back to the S₀ state or, through inter-system crossing to a triplet state (T₁).

The T₁ state can interact with other molecules via two pathways—both of which result in highly reactive species that together are termed ROS—and will subsequently oxidize other biomolecules (ie a virus) inactivating them.

Type 1—Direct electron transfer via a local substrate to form peroxide and hydroxyl radicals.

Type 2—Energy transfer to triplet (ground state) oxygen—producing the highly reactive singlet oxygen.

ROS although highly effective in disrupting biological system has a very short lifetime (a few microseconds), practically this means that it can only react with something that is very close to its point of formation (e.g., approximately 0.2 to 4 micrometers—depending on the environment), and is thus safe to use in a coating.

Photosensitizers come in an array of structures, including porphyrins, chlorins, phthalocyanines, xanthenes, isothiazines and many more—examples are shown in Figure B.

Examples of Photosensitizers for use in among others PS-ICF formulations are shown in FIG. 18.

Practically there are several other parameters that may come into play when choosing, or optimizing, a preferred photosensitizer for use in a particular field or application.

Light absorption—must be at a useful wavelength for the application, and the molecule should exhibit the strongest absorption cross-section possible (Epsilon >50,000).

Quantum yield—the amount of ROS (and thus T₁) produced per photon—this is a key measure of the efficiency of the photosensitizer.

Low photobleaching—i.e. the useful lifetime (cycles) of the photosensitizer.

Type 1 vs Type 2—it is generally accepted that for interaction with biological systems Type 2 (singlet oxygen production) is preferred.

“Stacking”—many photosensitizers self-associate as their concentration increases, inactivating the photosensitizer, thus lowering the production of ROS and efficacy.

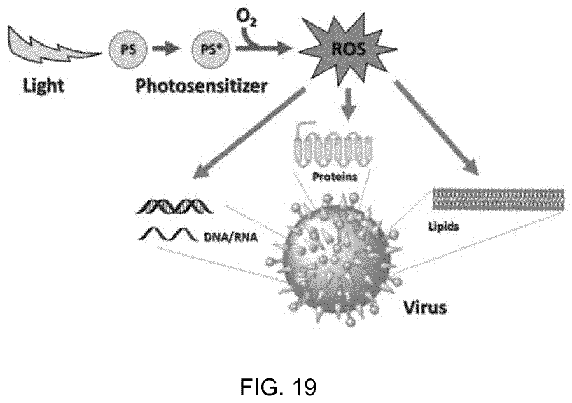

Generally for these processes Photodynamic Disinfection (“PDD”) has the presence of a photosensitizer, light, and oxygen to create ROS to inactivate a virus, or other pathogen. The non-specific destructive nature of PDT means that it can successfully interact with many parts of the virus as shown in FIG. 19.

### EXAMPLES

The following examples are provided to illustrate various embodiments of systems, processes, compositions, applications and materials of the present inventions. These examples are for illustrative purposes, may be prophetic, and should not be viewed as, and do not otherwise limit the scope of the present inventions.

### Example 1

Example of Product (PS(L)-NP-TA=IR700-8PEGA-Peptide).

A=Amine; MAL=maleimide; NHS=N-hydroxy succinimide.

The present invention utilizes the macropolymer 8-arm polyethylene glycol (8PEG-X), a TA (TA-Z), and a PS-L-Q, in any combination. The PS-L-Q is IR700-L-Q and its derivatives, the targeted tissue is a pathogen, and TA is a peptide. In the present specific case, the pathogen is COVID-19, and the corresponding TA is a fragment of ACE2 receptor (ACE2-F, IEEQAKTFLDKFNHEAEDLFYQS).

In one embodiment, TA-Z is conjugated directly with PS-L-Q, where PS-L-Q is IR700-NHS or IR700-MAL. IR700-NHS can be conjugated to the N-terminus of TA-Z or one of the lysine groups directly. IR700-MAL can be conjugated directly to TA-Z that has an added thiol group at the C or N-terminus (e.g. via an additional cysteine), or a lysine group that has been modified to be thiol terminated (e.g. cysteine). The product is a PS-TA conjugation.

In another embodiment, PS-TA-Z is covalently conjugated to 8PEG-X via a thiol-maleimide reaction, preferably X=MAL and Z=thiol; 8PEG-X may begin as a maleimide, or start as an amine that is converted to a MAL. Preferably, TA-Z=TA-cys, a cysteine terminated peptide. The product is PS-TA-8PEG.

Optionally, 8PEG-X may be conjugated with IR700-L-Q independently, and then further modified with IR700-TA. The product is PS-TA-8PEG-PS.

In the ideal embodiment, PS-L-Q is IR700-NHS or IR700-SH and 8PEG-X is A or MAL termination. IR700-NHS/SH is conjugated to 8PEG-X, yielding the form of 8PEGA-IR700 or 8PEGMAL-IR700 in a mol ratio that is less than 3:1 IR700:8PEG, but more than 1:1. IR700-8PEG-X is then conjugated to TA-Z, where preferably Z=thiol of cysteine.

ACE2-F and IR700-L-Q may be covalently conjugated with or without 8PEG-X in any combination, including, but not limited to: ACE2-F and IR700 conjugated as separate entities per arm; IR700 conjugated ACE2-F on 8PEG; and IR700 conjugated ACE2-F on IR700 conjugated 8PEG. In an embodiment the combination is to first conjugate IR700-L-Q to 8PEG-X and then attach the TA via 8PEG-X to ensure that at least 1 PS per 8PEG is present and that TA functionality is preserved by minimizing its modification.

### Example 2

An NP-PS nanocomposite composition for applying to, or use in, surfaces of materials to provide and active surface and active materials for use in PPR. The NP-PS can be any NP and any PS, including the NPs and PSs disclosed and taught in this specification.

The composition includes a liquid in which the NP-PS nanocomposite is contained. Generally, the NP-PS nanocomposition will be dispersed in this liquid, so that it remains in suspended in the liquid and does not agglomerate. The NP-PS nanocomposite remains dissolved, dispersed or suspended in the liquid and does not precipitate. Micelles/liposomes/vesicals, etc. can be used to solubilize the NP-PS nanocomposite. Preferably the NP-PS nanocomposite liquid combination forms a solution. The liquid can be water, an alcohol, and preferably can be a solution of materials that provides shelf life, better dispersion or spreading of the composition when used, and both of these.

In an embodiment the liquid is one or more of the compositions and materials taught and disclosed in U.S. Pat. No. 6,503,413, the entire disclosure of which is incorporated herein by reference.

The PS should be activated by light in the UV, visible and IR ranges. Preferably, the PS has an absorption peak, and a maximum absorption in a wavelength in the UV and visible wavelengths. The PS has a peak absorption, and a maximum absorption in a wavelength less than 600 nm, and from about 350 nm to about 600 nm. The PS has a peak absorption, and a maximum absorption in the near UV and blue wavelengths, e.g., less than about 550 nm, less than about 500 nm, and from 350 nm to 500 nm.

The NP-PS nanocomposite composition is packaged in a container that blocks, 80%, 90%, 99.9% and 100% of light from entering the container. The NP-PS nanocomposite composition is packaged in a container that blocks, 80%, 90%, 99.9% and 100% of light that is within 200 nm of the PS's peak absorption wavelength, that is within 100 nm of the PS's peak absorption wavelength, and that is within 50 nm of the PS's peak absorption wavelength.

The NP-PS nanocomposite composition can have a concentration of from about 1% NP-PS nanocomposite to about 80% NP-PS nanocomposite, from about 1% to about 10%, from about 5% to about 20%, more than 3%, more than 5%, more than 10%, more than 15%, more than 50% NP-PS. Generally, the NP-PS can have a concentration up to the point where the amount of NP-PS adversely effects the ability to apply the liquid to a surface or material, in particular apply the liquid to the surface or material in a uniform manner. In an embodiment the NP-PS nanocomposite composition is packaged in a container that blocks 90%.

### Example 3

An NP-PS nanocomposite composition for applying to, or use in, surfaces of materials to provide and active surface and active materials for use in PPR. The NP-PS can be any NP and any PS, including the NPs and PSs disclosed and taught in this specification.

The composition includes a liquid in which the NP-PS nanocomposite is contained. The liquid can be water, an alcohol, and preferably can be a solution of materials that provides shelf life, better dispersion or spreading of the composition when used, and both of these.

In an embodiment the liquid is one or more of the compositions and materials taught and disclosed in U.S. Pat. No. 6,503,413, the entire disclosure of which is incorporated herein by reference.

The composition has two, three or more PSs. Each having a different peak absorption wavelength. In this manner under various conditions of broad-spectrum ambient light, halogens, florescent, incandescent, LEDs, Sunlight, etc., the ROS will be produced and produced had an efficient and efficacious manner.

In a further embodiment of this multi-PS composition and materials, at least one of the PS, is not active, or has minimal absorption and activity, under visible light, and in particular under typical internal ambient lighting. Thus, the material treated with this NP-PS composition will have active-anti-pathogenic behavior, during exposure to ambient lighting, and them be place in a cleaning device under the non-visible wavelength or cleaning the material after use. (As noted in later Examples, this material can be retreated to provide second and third, etc. uses and cleanings of the material).

### Example 4

The NP-PS composite composition of Examples 2 and 3 are made without the use of an NP. In this manner the PS is not linked to an NP. The composition has one, two or three PS in a liquid. In these embodiments the liquid has dispersant and stabilization characteristics that permits the PS to remain active and effective after application to a material or surface. The PS remains dissolved, dispersed or suspended in the liquid and does not precipitate.

Micelles/liposomes/vesicals, etc. can be used to solubilize the PS nanocomposite. Preferably the PS nanocomposite liquid combination forms a solution.

### Example 5

The PS composition of Examples 2, 3, and 4 wherein the liquid is free of one or more of, and preferably all of: Formaldehyde, Bisphenol A, PVC (polyvinyl chloride), Triclocarban, Benzene, Flammable propellants (such as butane and propane), Organotins (DBT, TBT, MBT, DOT), PAHs (polycyclic aromatic hydrocarbons), Phthalates, Triclosan, Alkylphenols and alkylphenol ethoxylates and CFCs.

For each of the foregoing materials, in an embodiment, the composition has less than 1 ppm, less than 0.1 ppm, less than 0.001 ppm, and less than 0.0001 pm of any one of the foregoing materials.

For each of the foregoing materials, in an embodiment, the composition has less than 1 ppm, less than 0.1 ppm, less than 0.001 ppm, and less than 0.0001 pm of each of the foregoing materials.

For each of the foregoing materials, in an embodiment, the composition has less than 1 ppm, less than 0.1 ppm, less than 0.001 ppm, and less than 0.0001 pm of all of the foregoing materials in aggregate (e.g., total all of the foregoing materials).

### Example 6

The PS composition of Examples 2, 3, 4 and 5, wherein the liquid has one or more and preferably all of: Water, Nitrogen, Cyclodextrin, Didecyl Dimethyl Ammonium Chloride, Modified Polydimethicone, Alcohol, Hydrogenated Caster Oil, Maleic Acid, Dialky Sodium Sulfosuccinate, Sodium Citrate, Dithyllene Glycol, Benzisothiazolinone, Polyamines, Petrolatum Wax, Paraffin Wax, and Soy Wax.

### Example 7

The PS composition of Examples 2, 3, 4, 5 and 6, wherein the liquid and the composition are configured for application to a material or surface by spraying the composition onto a target material.

The target materials can be a fiber (natural or synthetic), paper (paper products), plastics, woven fabric, non-woven fabric, fur, leather, a hard surface, glass surface, metal surface, stone surface, porous surfaces, a formed product, surface of a composite, a composite material or web, paint surface, thermally bonded surface, coating surface, a sheet of material, a roll of material, a mask, a gown, a coat, gloves, surfaces on a transportation device (e.g., trucks, cars, planes, boats, buses, etc.), clothing, PPE, masks, face protection, counter tops, tables, desks, seats, medical equipment surfaces, etc.

These treated materials are active materials and PPRs.

### Example 8

The PS composition of Examples 2, 3, 4, 5 and 6, wherein the liquid and the composition are configured for application to a material or surface by liquid application, such as rollers, presses, immersion, flotation. A method of apply the compositions of Examples 2, 3, 4, 5, and 6 by spraying the composition onto a target material.

The target materials can be a fiber (natural or synthetic), paper (paper products), plastics, woven fabric, non-woven fabric, fur, leather, a hard surface, glass surface, metal surface, stone surface, porous surfaces, a formed product, a composite material or web, a sheet of material, a roll of material, a mask, a gown, a coat, gloves, surfaces on a transportation device (e.g., trucks, cars, planes, boats, buses, etc.), clothing, PPE, masks, face protection, counter tops, tables, desks, seats, medical equipment surfaces, etc.

These treated materials are active materials and PPRs.

### Example 9

The PS composition of Examples 2, 3, 4, 5 and 6, wherein the liquid and the composition are configured for application to a material or surface by aerosolization or as an aerosol. A method of apply the compositions of Examples 2, 3, 4, 5, and 6 by treating with a target material with an aerosol of the composition,

The target materials can be a fiber (natural or synthetic), paper (paper products), plastics, woven fabric, non-woven fabric, fur, leather, a hard surface, glass surface, metal surface, stone surface, porous surfaces, a formed product, a composite material or web, a sheet of material, a roll of material, a mask, a gown, a coat, gloves, surfaces on a transportation device (e.g., trucks, cars, planes, boats, buses, etc.), clothing, PPE, masks, face protection, counter tops, tables, desks, seats, medical equipment surfaces, etc.

These treated materials are active materials and PPRs.

### Example 10

The PS composition of Examples 2, 3, 4, 5 and 6, wherein the liquid and the composition are configured for application into a manufacturing process for a web of material, a fiber, a sheet of material, a composite material, a molded material, a formed material, and structures or devices made from these. In this embodiment the compositions are added into a point in the manufacturing process and thus provide an active material. In such applications care should be taken to control the PS to light, and in particular light in the wavelength where the PS has peak absorption, during the manufacturing process up to and including packaging.

The materials, where the PS composition is added into the manufacturing process, can be a fiber (natural or synthetic), paper (paper products), plastics, woven fabric, non-woven fabric, fur, leather, a hard surface, glass surface, metal surface, stone surface, porous surfaces, a formed product, a composite material or web, a sheet of material, a roll of material, a mask, a gown, a coat, gloves, surfaces on a transportation device (e.g., trucks, cars, planes, boats, buses, etc.), clothing, PPE, masks, face protection, counter tops, tables, desks, seats, medical equipment surfaces, etc.

In this embodiment the NP-PS composite, can in embodiments be applied without a liquid, e.g., a lyophilized material. Although, preferably the NP-PS is in a liquid when added to or used in the manufacturing process.

These treated materials are active materials and PPRs.

### Example 11

One or more of a PS composition, a NP-PS composition, and the PS compositions of Examples 2, 3, 4, 5 and 6, is applied to a non-woven fabric. Thus, the non-woven fabric is treated with the NP-PS composition of Example 2. The treated material is an active material and a PPR.

The NP-PS is distributed, preferably uniformly, on the surface and in this manner can be envisioned as forming a layer, preferably a uniform layer or coating, on the surface of the fabric. Upon exposure to light, and preferably light including light with the wavelength of the peak absorption for the PS, has active anti-pathogenic properties (i.e., it generates ROS) for at least 5 minutes, for at least 10 minutes, for at least 30 minutes, for about 5 minutes to about 4 hours, for about 1 hours to about 12 hours, and longer.

The treated fabric is packaged in a package that prevents activation of the PS, prior to use.

The treated fabric can be a final product, such as PPE, cover, hat, etc., or it can be sheet or roll material, that is stored and later used to make a final product.

These treated materials are active materials and PPRs.

### Example 12

One or more of a PS composition, a NP-PS composition, and the PS compositions of Examples 2, 3, 4, 5 and 6, is applied to a woven fabric. Thus, the woven fabric is treated with the NP-PS composition of Example 2.

The NP-PS forms a layer, preferably a uniform layer or coating, on the surface of the fabric. Upon exposure to light, and preferably light including light with the wavelength of the peak absorption for the PS, has active anti-pathogenic properties (i.e., it generates ROS) for at least 5 minutes, for at least 10 minutes, for at least 30 minutes, from about 5 minutes to about 4 hours, from about 1 hours to about 12 hours, and longer. Preferably, the ROS generation is continuous during these periods, and more preferably during this period is uniform.

The treated fabric is packaged in a package that prevents activation of the PS, prior to use.

The treated fabric can be a final product, such as PPE, cover, hat, etc., or it can be sheet or roll material, that is stored and later used to make a final product.

These treated materials are active materials and PPRs.

### Example 13

One or more of a PS composition, a NP-PS composition, and the PS compositions of Examples 2, 3, 4, 5 and 6, is applied to a paper material. Thus, the paper material is treated with the NP-PS composition of Example 2.

The NP-PS forms a layer, preferably a uniform layer or coating, on the surface of the paper material. Upon exposure to light, and preferably light including light with the wavelength of the peak absorption for the PS, has active anti-pathogenic properties (i.e., it generates ROS) for at least 5 minutes, for at least 10 minutes, for at least 30 minutes, for about 5 minutes to about 4 hours, for about 1 hours to about 12 hours, and longer. Preferably, the ROS generation is continuous during these periods, and more preferably during this period is uniform.

The treated material is packaged in a package that prevents activation of the PS, prior to use.

The treated material can be a final product, such as PPE, cover, hat, etc., or it can be sheet or roll material, that is stored and later used to make a final product.

These treated materials are active materials and PPRs.

### Example 14

One or more of a PS composition, a NP-PS composition, and the PS compositions of Examples 2, 3, 4, 5 and 6, is applied to a solid surface. The solid surface can be any surface, such as a counter top, a surface of a medical device, equipment or infrastructure (such as, an MRI, dialysis machine, imaging devices, CAT scans, beds, will chairs, floors, walls, ndesks, nursing stations, elevators, etc.), surface of manufacturing facilities (such as, meat processors, automotive manufactures, food processors, etc.), surfaces in kitchens, tables, surfaces in public transit, surface in airports and planes, surfaces in ships, surfaces in amusement parks, surfaces in public venues, etc. Thus, the solid surface is treated with the NP-PS composition of Example 2.

The NP-PS forms a layer, preferably a uniform layer or coating, on the surface of the paper material. Upon exposure to light, and preferably light including light with the wavelength of the peak absorption for the PS, has active anti-pathogenic properties (i.e., it generates ROS) for at least 5 minutes, for at least 10 minutes, for at least 30 minutes, from about 5 minutes to about 4 hours, from about 1 hours to about 12 hours, and longer. Preferably, the ROS generation is continuous during these periods, and more preferably during this period is uniform.

The treated material is packaged in a package that prevents activation of the PS, prior to use.

The treated material can be a final product, such as PPE, cover, hat, etc., or it can be sheet or roll material, that is stored and later used to make a final product.

These treated materials are active materials and PPRs.

### Example 15

Preferably during all manufacturing activities, treatment of sheet or roll materials for later use, treatment of products, for later use, the treatment and storage are done under optical conditions where the light is far removed from the wavelength that activates, and preferably is the peak activation wavelength for the PS. Thus, for example, in treating a web of fabric that is being produced into roll form, the section of the apparatus where the PS-NP composition is applied to the web, and thereafter, to the extent light is present, should be a wavelength that is at least 100 nm, at least 200 nm, at least 300 nm away from the beak. For example, a NP-PS having a peak absorption below 500 nm can be manufactured in light having a wavelength of greater than 650 nm, and preferably greater 750 to 780 nm.

### Example 16

Products, materials, surface, including products intended for single use, can be treated with one or more of a PS composition, a NP-PS composition, and the PS compositions of Examples 2, 3, 4, 5 and 6, just prior to use, during use, and after use. In this manner the treated material or product would provide an active anti-photogenic material. Further, any pathogens on the material or product, prior to treatment will be destroyed by the ROS generated by the PS, in which manner the material can be disinfected.

For products that are intended to provide, or configured to provide a barrier to, filtration of, and both, a pathogen, such as bacteria or viruses, the treated material or product becomes an active filter, upon exposure to light. In this manner the material or product is generating ROS and activity killing, destroying or rendering inert the pathogens. This will greatly increase the filtration ability and safety of the product.

During use, and for example, reuse of a product labeled or identified as single use, the treatment can be repeatedly applied and reapplied. In this manner the treated products active barrier can be maintained for extended periods to time, e.g., more than 1 hour, more than 2 hours, more than 12 hours, more than 24 hours.

### Example 17

An illumination and disinfectant chamber, for decontaminating products, materials, and the surfaces of devices and equipment. The chamber has light generation devices, preferably that generate a light field that will enter any and all cracks, folds, corners, etc. of the material or product to be decontaminated. Preferably, the light in the chamber is of a wavelength that is the optimum wavelength to activate the PS and generate ROS. The light source can be LEDs, Lasers, coherent light, scanned lasers, etc. Sufficient energy should be applied to activate the die and generate the ROS.

As the PS generates ROS from ambient, in situ or nearby oxygen sources, the chamber can have a supplemental oxygen flow added to the chamber. Preferably the additional oxygen is kept at or below a level with fire or explosive risks are present.

The products or materials are treated with a PS composition, a NP-PS composition, and the PS compositions of Examples 2, 3, 4, 5 and 6, and then the treated products or materials are placed in the chamber and illuminated.

The materials or products can be illuminated for 5 mins to hours to several hours. The materials can be illuminated until all pathogens are rendered inert. For example, such that the illuminated material or product has less than 0.001 ppm active pathogens, less than 0.0001 ppm active pathogens, less than 0.00001 ppm active pathogens, less than 0.000001 ppm active pathogens, and zero active pathogens on their surfaces.

The disinfected materials and product can then have a PS treatment applied to them, placed in a light blocking container, so that they are ready for the next use, and will provide an active surface and PPR.

### Example 19

A method of disinfection a large medical device, such as an x-ray machine, a CAT scanner, an MRI, and other surgical or diagnostic devices. The surfaces of the device are treated with one or more of a PS composition, a NP-PS composition, and the PS compositions of Examples 2, 3, 4, 5 and 6. The device, and in particular all surfaces are illuminated with light, preferably having light in the wavelength of the peak absorption of the PS(s). The light can be delivered by lamps, LEDs, lasers, optical fibers and combinations and variations of these.

In this embodiment the surfaces of the device can be disinfected in less than 30 minutes of illumination, in less than 15 minutes of illumination, and in less than 5 minutes of illumination.

The liquid should be safe for application to surfaces that may have electronic components associated with the, such as switches and sensors. Further, and preferably, the liquid should be such that it evaporates, or is easily wiped away, and does not need further cleaning.

### Example 20

NP-PS system in pH controlled water/alcohol systems from 100% water to 80/20 water/alcohol produce ROS upon exposure to activation light. The NP-PS system is stable and when exposed to light continuously produces reactive oxygen species that are active against pathogens.

### Example 21

The NP-PS is be dried, e.g., to a powder, for safe storage and will quickly and easily re-disperse in any of the above liquids disclose and taught in this specification with full efficacy.

### Example 22

Application to a PPE mask. Desired coverage is 1 microgram/cm² (of PS). One “spray” is about 0.1-0.2 ml. Area of the mask is 18 sq in (˜100 cm²). NP-PS that is about 40 k, Use 3 sprays=˜0.5 ml (each) of a 2 mg/ml a 2% NP-PS solution.

### Example 23

In an embodiment there is provided a formulation that provided PDD on a surface (e.g., hard surface, woven fabric or non-woven fabric) in the absence of moisture, e.g., a dry surface. Thus, PDD is achieved when the surface has less than 5%, less than 2%, less than 1%, and less than 0.5% moisture. Embodiments will also function providing PDD, when greater amounts of moisture are present.

### Example 24

In an embodiment PDD is achieved on a dry surface, with ambient lighting. This capability to provided PDD in the absence of a culture medium, e.g., on a “dry” surface and with illumination from ambient lighting rather than distinct, controlled single wavelength illumination, provides advantages, to the formulations use in a wide variety of circumstances and environments. Such a formulation may be: A formulation for simple at point application—requiring no specialized knowledge or technique. Immobilizing a high concentration of the photosensitizer on a variety of surfaces. Maintaining a high production of ROS over a defined period—and delivering at least a log 3 reduction in active viral load. Functioning under a variety of lighting conditions.

Thus, PDD is achieved when the surface has less than 5%, less than 2%, less than 1%, and less than 0.5% moisture. Embodiments will also function providing PDD, when greater amounts of moisture are present.

### Example. 25

A method of placing a high concentration of active photosensitizer onto a surface in a safe and simple “spray-on” formulation. Formulated this solution with materials that have the correct (existing) regulatory profile for the intended use.

### Example 26

A water-based formulation—that could be formulated as either a concentrate or a ready to use solution. In the case of the concentrate all that would be needed would be dilution with water. This solution is sprayed onto the desired surface (˜0.3 ml for a face mask), this will evenly deposit, rapidly dry and immobilize an effective concentration of photosensitizer on the material avoiding “Stacking” and ensuring a highly efficient production of ROS under common lighting conditions.

### Example 27

A PS-ICF formulation, having one or more of the following PS

methylene blue (CAS #61-73-4)

Rose Bengal (CAS #632-69-9)

Riboflavin (CAS #83-88-5)

Toluidine Blue (CAS #92-31-9)

Eosin Blue (CAS #16423-68-0)

### Example 28

A formulation having one or more PS, e.g., Example 27, and including the following in active ingredients:

A carrier molecule that holds/immobilizes the photosensitizer on the target surface. This molecule carries and delivers the photosensitizer to the surface, and upon drying of the coating, ensures optimum coverage, orientation, and catalytic effect. The carrier may covalently bound, or otherwise associated with the photosensitizer.

A hydroxypropyl-beta-cyclodextrin (HPBCD) that forms an “inclusion complex” (shown left) with the photosensitizer e.g., methylene blue. Beta-cyclodextrin (BD) is a small cyclic polymer of glucose (six residues.

A surfactant/wetting agent—to promote the even spreading (wetting) and adhesion of the coating on the surface. For example polysiloxane non-ionic surfactants. Preferably these can be drawn from existing and approved materials

Alcohol (Ethanol)—to aid in overall solubility of components and promote the rapid drying of the coating.

Buffers/Preservatives—to maintain the formulation once prepared. Preferably these can be drawn from existing and approved materials.

### Example 29

A formulation having one or more PS, e.g., Example 27, and including the following in active ingredients:

A carrier molecule that holds/immobilizes the photosensitizer on the target surface. This molecule carries and delivers the photosensitizer to the surface, and upon drying of the coating, ensures optimum coverage, orientation, and catalytic effect. The carrier may covalently bound, or otherwise associated with the photosensitizer.

A PS, e.g., methylene blue, to a multi-arm polyethylene glycol (PEG) molecule (below).

A surfactant/wetting agent—to promote the even spreading (wetting) and adhesion of the coating on the surface. For example polysiloxane non-ionic surfactants. Preferably these can be drawn from existing and approved materials

Alcohol (Ethanol)—to aid in overall solubility of components and promote the rapid drying of the coating.

Buffers/Preservatives—to maintain the formulation once prepared. Preferably these can be drawn from existing and approved materials.

### Example 29

Derivation of any PEG (linear, or branched of any molecular weight) to place an “inclusion complex former” (ICF) at the terminus of each “arm/chain”.

In an embodiment any polymeric NP, cross linked or otherwise—upon which the ICF can be conjugated, without loss of its properties.

Some advantages for this Example are:

Makes the ICF fully water compatible (in case it is not)
Allows binding not only “standard” PS's to the NP but also those which:
Cannot be chemically conjugated to the NP without loss of PD performance
Are so hydrophobic (eg Verteporfin) that the NP-PS is difficult to form and loses its solubility
Can enhance the triplet state of the PS (longer lifetime) making it a more effective ROS producer
May protect the PS from photobleaching

For PEG embodiments:

### Preferred is 8 PEG, 20-40 kDa

Loading can be from 1 to 8 ICF's—but prefer 2-5
Can be of the form:
NP-ICF alone
TA-NP-ICF (for this thought, simply take our comments on TA-NP-PS and subs ICF for PS)

Chemistry/Formulating

Any of Tables 2A, 3A and 4A can be used. For the formation of the NP-ICF—take the tables that of the specification and formation of the NP-PS and simply substitute the ICF for the PS the ICF can be added to the NP as the ICF alone OR the IFC/PS inclusion complex—preferably put just the ICF on first.

When forming the TA-NP-ICF/PS the PS can in an embodiment be added to the TA-NP-ICF. In an embodiment the TA can be added to the NP-ICF/PS.

ICF's

All the cyclodextrins (alpha/beta/gamma and their derivatives)

Making the ICF/Inclusion Complex

Dissolving the NP-ICF, or TA-NP-ICF, in excess (at least 10×ICF to PS—preferentially 50-100) and the PS in a compatible solvent (buffered water, water/alcohol mixtures specifically) and allowing to equilibrate for a period of time, 30-60 mins, usually at room temperature and then removing the solvent to produce a lyophilized solid for final use

Preferrably, excess of ICF over PS should be used to ensure that essentially all the PS is complexed (ie there is essentially no free dye)

In embodiments this process can make mixtures of PS by associating the ICF's with more that one PS.

Further Advantages

With this structure and method of NP-ICF or TA-NP-ICF we can essentially place any molecule that can form an “inclusion” complex form mixtures of these Examples

2 or more PS's
A PS and a drug active towards the desired site (ie Photo and chemotherapy in one)
Two synergistic drugs—no PS
Solubilizing highly hydrophobic drugs
Applications in pesticides and herbicides

### Example 30

A spray bottle containing the formulations of Examples 27, 28, 29, or any of the other formulations of the Examples.

### Example 31

An item of PPE coated with, or containing, the formulations of Examples 27, 28, 29, or any of the other formulations of the Examples.

### Example 32

The method of coating an item of PPE the formulations of Examples 27, 28, 29, or any of the other formulations of the Examples.

### Example 33

The formulations and embodiments of Examples 1 to 29, and any PS formulations and embodiments in this specification, are applied to, used to treat or incorporated into a covering, e.g. film, for a surface. In this manner there is provided a covering, that preferably has one side having a removably adhesive surface. The covering has the PS formulation contained on, contained within, or otherwise associated with it. Thus, providing an active covering against pathogens for articles and surfaces.

In embodiments the active cover can be a transparent film, which could have a color if desired, or could be opaque. The film is than placed on, preferably removably adhered to a surface or an article.

In an embodiment several films are attached, as a stack, or layers of multiple films, that can be removed to expose a fresh film below. In this multilayer embodiment, the films should have an inner layer, or lower surface that is black or non-transparent to the wavelength of the activation light, or have layers interspersed between them to block the activation light wavelength, and thus, prevent activation of the lower layers, until they are exposed or become the top layer.

In embodiments these covering films can be applied to graphic user interfaces (GUI) on any device or system. They can be applied key pads for any device or system. They can be applied to any table top, hand rail, control panel, or counter top. Thus, for example, these covering films can be applied to:

Airport Kiosks

Airplane (back of seat) trays

Airplane TVs (back of seats)

Airplane Cockpit touchscreens

MTA/CTA Subway Kiosks

iPads/iPhones

Microsoft Surfaces

Android Phones

Keyboards

Pelaton Screen

Gym Equipment (public)

Elevators

Public Bathrooms

Reception desks/Kitchen Surfaces (office)/Conference Tables

Bar Surfaces

Front Door Handles/Rotary Handles and windows

Subway handrails

Subway seats

Retail Cash Registers

Handles within dressing rooms (retail)

Rental Cars (steering wheels)

Food Wrapping

Playgrounds/Park Benches

Taxis/Uber/Lyft

Restaurant Tables/Seats

Menus

All touchscreens

Industrial coverings (safety)

Wrapping for “sanitized” products (i.e. hairdresser products)

Laundry Mats lids and handles

Dry cleaning plastic

Elevators and escalators

Non-aqueous systems as these will have merit and value in manufacturing of items where ease of application and drying rate are very important—these formulations may have value in the consumer end use markets as well.
This refers to the in-situ treatment/production of articles such that:
   Said articles when exposed to a suitable light source (daylight or ambient lighting) actively disinfect their (own) surface and other articles placed close to them
   But may also retain anti-microbial activity in the dark through a combination of other anti-microbial agents
      Certain anti-microbials may have a synergistic effect, where PS (ROS)+antimicrobial action is more than 1+1=2
         QAC's and active oxygen species appear act synergistically in the breakdown of biofilms and certain pathogens
   Protect the handler/user from infection by pathogens transmitted via contact with said surface
These may be applied to one time/limited time use product
Said items may be recycled into the usual stream without the need for separation or special treatment
Said items provide for a continually disinfected surface for the lifetime of use
is applicable for items made with a wide variety of materials, not limited to but including papers, cardboards, wood, metal, plastic (all types), ceramics, glass and composites of all the above.
Examples for use may be
   Packaging materials—boxes, wrapping paper or plastic
   Plastic films or bags for the protection of other products
   Mats, coverings etc to protect work surfaces, tables etc
Through the “adjacent disinfection mechanism” of ROS items may also provide temporary but effective anti-microbial protection to items place on the surface of such items—silverware, phones, keys, coins, surgical items (in healthcare environment)

Basic Concept
A range of formulations—optimized for the surface in question—that when applied provide for a coating that in the presence of light (daylight or ambient) continuously generate an effective flux of reactive oxygen species that inactivate substantially all pathogens present on, or in close proximity to the surface

Basic Formulation
A range of formulations—optimized for the surface in question—that when applied provide for a coating that in the presence of light (daylight or ambient) continuously generate an effective flux of reactive oxygen species that inactivate substantially all pathogens present on, or in close proximity to the surface

Minimally Contains
A photosensitizer (eg methylene blue or any other or mixture thereof)
An inclusion complex former (ICF)
A non-aqueous solvent system capable of dissolving substantially all of the components of the system (can be protic or aprotic solvents)—drawn from, but not limited to
   Alcohols
   Ethers
   Dimethyl formamide (DMF)
   Acetonitrile
   DMSO

Can Additionally Contain
Non-ionic surfactants
Wetting and filming agents
Cationic surfactants
Anionic surfactants
Quaternary ammonium compounds
Other antimicrobial reagents
Polymers
Odor controlling agents
Scents/fragrances
Colorants
A nanoparticle conjugates to the Photosensitizer
Any other material used in the formation of coatings and surface treatments
Formulations are applied either during the manufacturing process of the article or at point of use
Formulation is applied through standard processes including but not limited to
   Spraying
   Rolling
   Flooding (doctor blade)
   Brushing
   Laminating
   Painting
   Aerosol
Is applied to deliver an effective concentration of “Active” PS to the surface (addition of the inclusion complex former optimizes this by deliver more of the ‘active monomeric’ form of the photosensitizer to the surface

Formulation should Deliver—Ie it is Designed to Deposit to the Target Surface
0.05 ug to 1 mg/cm2 of the active PS to the surface
Most preferred to be in the 1-2 ug/cm2 (for MB)
For all PS's this would be a preferred range of 3 nanomoles/cm2 to 6 nanomoles/cm2

Example Formulation

| | |
|---|---|
| Methylene blue | 0.005% w/v (.05 g/L) |
| 2 Hydroxypropyl beta-cyclodextrin | .375% w/v (3.75 g/L) |
| Ethanol | balance to 100% |
| With or without | |
| Film former (eg Silwet L77) | 0.2% w/v |
| A quaternary ammonium salt (quat) | 1.0% w/v |

Eg Didecyl Dimethyl ammonium chloride (DDAC)

### EMBODIMENTS

Coated Rolls of paper (one or two sides) used to cover, laminate, wrap items, or as “Stand-alone” product to protect a surface (eg mats)
Coated rolls of cardboard stock (one or two sides) for use in cartons or corrugated boxes or other corrugated products
Coated rolls of solid plastic (polyethylene, polypropylene, polyamide, polyester . . . ) film (one or two sides) for use to laminate/cover other materials (eg cardboard, paper, work surfaces, ceramics, metals and composites thereof)
Coated plastic foamed roll/sheet products (eg polystyrene, poly olefins)—cut to size and used as covering and mats
Metal cans for packaging
Concentrates, finished formulations in tanks, bottles, spray cans—to apply coating at point of use—ie to an untreated cardboard boxes, metal containers—applied through any common coating process.

Targeted Uses
Boxes
Work surfaces
Healthcare work surfaces/surgical theaters
Paper coverings
Metal cans
Soft materials/fabrics

Statement 1: A material comprising paper fibers, selected from the group of liner board, liner, medium, corrugated, corrugated containers, boxes, corrugated boxes, sheet material, and corrugated sheet material having a surface having a stable, photodynamic disinfection composition said composition comprising:
(a) a polyalkyleneoxide polysiloxane having the formula:

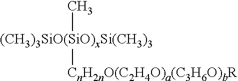

wherein x is from about 1 to about 8; n is from about 3 to about 4; a is from about 1 to about 15; b is from about 0 to about 14; a+b is from about 5 to about 15; and R is selected from the group consisting of hydrogen, an alkyl group having from about 1 to about 4 carbon atoms, and an acetyl group; and wherein said polyalkylene polysiloxane has a molecular weight of less than about 1,000;
(b) a buffering agent; wherein said buffering agent has at least one pKa value and/or ply, value of from about 4 to about 10;
(c) an aqueous carrier;
(d) a photosensitizer associated with an inclusion complex former;
(e) wherein said composition has a pH of from about 4 to about 10.

Statement 2. The compositions of any preceding statement, wherein the photosensitizer is selected from the group consisting of methylene blue (CAS #61-73-4), Rose Bengal (CAS #632-69-9), Riboflavin (CAS #83-88-5), Toluidine Blue (CAS #92-31-9) and Eosin Blue (CAS #16423-68-0).

Statement 3. The compositions of any preceding statement, wherein the photosensitizer is selected from the group consisting of Curcumin, Verteporfin, Erythrosin B, New MB, and Eosin Y, Erythrosine.

Statement 4. The compositions of any preceding statement, wherein the photosensitizer is selected from the group consisting of PHOTOFRIM, Photochlor (CAS #149402-51-7), IR700 Chlorin e6, Protoporphyrin IX, NPe6PHCurcumin, Verteporfin, Erythrosin B, New MB, Eosin Y, and Erythrosine.

Statement 5. The compositions of any preceding statement, wherein the inclusion complex former is selected from the group consisting of cyclodextrins, unsubstituted cyclodextrins, alpha-cyclodextrin, beta-cyclodextrin, gamma-cyclodextrin, calixarenes, cryptands and crown ethers and derivatives of each of these.

Statement 6. The compositions of any preceding statement, wherein the inclusion complex is covalently bonded to a nanoparticle.

Statement 7. The compositions of any preceding statement, wherein the nanoparticle is selected from the group of PEG, 8-PEGA, and PAA.

Statement 8. The compositions of any preceding statement, wherein the composition comprises a targeting agent.

Statement 9. The compositions of any preceding statement, wherein the photosensitizer is associated with the inclusion complex former by Van der Waals forces.

Statement 10. The compositions of any preceding statement, wherein said composition further comprises a cationic surfactant.

Statement 11. The compositions of any preceding statement, wherein said aqueous carrier comprises water and less than about 20% alcohol, wherein said alcohol is a monohydric or polyhydric alcohol.

Statement 12. The compositions of any preceding statement, wherein said composition further comprises a perfume.

Statement 13. The compositions of any preceding statement, wherein said composition further comprises a supplemental wrinkle control agent.

Statement 14. The compositions of any preceding statement, wherein said supplemental wrinkle control agent is selected from the group consisting of fiber lubricants, shape retention polymers, hydrophilic plasticizers, lithium salts, and mixtures thereof.

Statement 15. The compositions of any preceding statement, wherein said composition further comprises an additional co-surfactant selected from the group consisting of nonionic surfactants, anionic surfactants, zwitterionic surfactants, fluorocarbon surfactants, and mixtures thereof.

Statement 16. A material comprising paper fibers, selected from the group of liner board, liner, medium, corrugated, corrugated containers, boxes, corrugated boxes, sheet material, and corrugated sheet material having a surface having a stable, photodynamic disinfection composition said composition comprising:
(a) a polyalkyleneoxide polysiloxane having the formula:

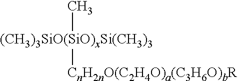

wherein x is from about 1 to about 8; n is from about 3 to about 4; a is from about 1 to about 15; b is from about 0 to about 14; a+b is from about 5 to about 15; and R is selected from the group consisting of hydrogen, an alkyl group having from about 1 to about 4 carbon atoms, and an acetyl group; and wherein said polyalkylene polysiloxane has a molecular weight of less than about 1,000;
(b) a cationic surfactant;
(c) a buffering agent; wherein said buffering agent has at least one pKa value and/or ply, value of from about 4 to about 10;
(d) aqueous carrier;
(e) a photosensitizer associated with an inclusion complex former; and,
(f) wherein said composition has a pH of from about 4 to about 10.
Statement 17. The compositions of any preceding statement, wherein the photosensitizer is selected from the group consisting of methylene blue (CAS #61-73-4), Rose Bengal (CAS #632-69-9), Riboflavin (CAS #83-88-5), Toluidine Blue (CAS #92-31-9) and Eosin Blue (CAS #16423-68-0).

Statement 18. The compositions of any preceding statement, wherein the photosensitizer is selected from the group consisting of Curcumin, Verteporfin, Erythrosin B, New MB, and Eosin Y, Erythrosine.

Statement 19. The compositions of any preceding statement, wherein the photosensitizer is selected from the group consisting of PHOTOFRIM, Photochlor (CAS #149402-51-7), IR700 Chlorin e6, Protoporphyrin IX, NPe6PHCurcumin, Verteporfin, Erythrosin B, New MB, Eosin Y, and Erythrosine.

Statement 20. The compositions of any preceding statement, wherein the inclusion complex is selected from the group consisting of cyclodextrins, unsubstituted cyclodextrins, alpha-cyclodextrin, beta-cyclodextrin, gamma-cyclodextrin, calixarenes, cryptands and crown ethers and derivatives of each of these.

Statement 21. The compositions of any preceding statement, wherein the inclusion complex former is covalently bonded to a nanoparticle.

Statement 22. The compositions of any preceding statement, wherein the nanoparticle is selected from the group of PEG, 8-PEGA, and PAA.

Statement 23. The compositions of any preceding statement, wherein the composition comprises a targeting agent.

Statement 24. The compositions of any preceding statement, wherein the photosensitizer is associated with the inclusion complex former by Van der Waals forces.

Statement 25. The compositions of any preceding statement, wherein said composition further comprises a perfume.

Statement 26. The compositions of any preceding statement, wherein said composition further comprises a supplemental wrinkle control agent.
Statement 27. A method of forming a material comprising paper fibers, selected from the group of liner board, liner, medium, corrugated, corrugated containers, boxes, corrugated boxes, sheet material, and corrugated sheet material having a surface having stable photodynamic disinfection composition, by applying said composition to the material, wherein the composition comprises:
(a) a first liquid; and,
(b) an inclusion complex comprising a photosensitizer associated with an inclusion complex former;
(c) wherein when applied to a surface and upon exposure to light the photosensitizer is configured to generate ROS from ambient oxygen;
(d) whereby pathogens adjacent to the surface are killed.

Statement 28. The compositions of any preceding statement, wherein the light is selected from ambient light, sun light, visible light.

Statement 29. The compositions of any preceding statement, wherein the first liquid is a surfactant.

Statement 30. The compositions of any preceding statement, further comprising a buffering agent.

Statement 31. The compositions of any preceding statement, further comprising an aqueous carrier.

Statement 32. The compositions of any preceding statement, wherein said composition has a pH of from about 4 to about 10

Statement 33. The compositions of any preceding statement, further comprising an alcohol.

Statement 34. The compositions of any preceding statement, further comprising an ethanol.

Statement 35. The compositions of any preceding statement, wherein the photosensitizer is selected from the group consisting of methylene blue (CAS #61-73-4), Rose Bengal (CAS #632-69-9), Riboflavin (CAS #83-88-5), Toluidine Blue (CAS #92-31-9) and Eosin Blue (CAS #16423-68-0).

Statement 36. The compositions of any preceding statement, wherein the photosensitizer is selected from the group consisting of Curcumin, Verteporfin, Erythrosin B, New MB, and Eosin Y, Erythrosine.

Statement 37. The compositions of any preceding statement, wherein the photosensitizer is selected from the group consisting of PHOTOFRIM, Photochlor (CAS #149402-51-7), IR700 Chlorin e6, Protoporphyrin IX, NPe6PHCurcumin, Verteporfin, Erythrosin B, New MB, Eosin Y, and Erythrosine.

Statement 38. The compositions of any preceding statement, wherein the inclusion complex former is selected from the group consisting of cyclodextrins, unsubstituted cyclodextrins, alpha-cyclodextrin, beta-cyclodextrin, gamma-cyclodextrin calixarenes, cryptands and crown ethers and derivatives of each of these.

Statement 39. The compositions of any preceding statement, wherein the inclusion complex is covalently bonded to a nanoparticle.

Statement 40. The compositions of any preceding statement, wherein the nanoparticle is selected from the group of PEG, 8-PEGA, and PAA.

Statement 41. The compositions of any preceding statement, wherein the composition comprises a targeting agent.

Statement 42. The compositions of any preceding statement, wherein the photosensitizer is associated with the inclusion complex former by Van der Waals forces.

Statement 43. The compositions of any preceding statement, wherein the photosensitizer is configured to generate ROS for about 4 hours to about 96 hours.

Statement 44. The compositions of any preceding statement, wherein the photosensitizer is configured to generate ROS for at least 24 hours.

Statement 45. The compositions of any preceding statement, wherein the photosensitizer is configured to generate ROS for at least 48 hours.

Statement 46. The compositions of any preceding statement, wherein the photosensitizer is configured to generate ROS for at least 96 hours.

Statement 47. A spray bottle comprising any of the compositions of any preceding statement.

Statement 48. A method of making a surface of an article an active surface for killing pathogens, wherein the article is selected from the group of liner board, liner, medium, corrugated, corrugated containers, boxes, corrugated boxes, sheet material, and corrugated sheet material the method comprising:

applying any of any of the compositions of any preceding statement to the article;

whereby a surface of the article is coated with the component comprising the photosensitizer associated with the inclusion complex former;

thereby providing the surface with photodynamic disinfectant properties.

Statement 49. The method of any preceding statement, wherein the surface is selected from the group consisting of hard surfaces, fibers, woven fabrics, non-woven fabrics, natural fibers, synthetic fibers, films, natural surfaces, synthetic surfaces, plastics, stone, and metal.

Statement 50. The methods of any preceding statement, wherein the article is a PPE.

Statement 51. The methods of any preceding statement, wherein the pathogen is SARS-CoV-2.

Statement 52. The methods of any of any preceding statement, wherein the pathogen is selected from the group consisting of influenza viruses, corona viruses, SARS-CoV-2 (causing COVID-19), Ebola, HIV, SARS, H1N1 and MRSA, as well as, Campylobacter, Clostridium Perfringens, E. coli, Listeria, Norovirus, Salmonella, Bacillus cereus, Botulism, Hepatitis A, Shigella, Staphylococcus aureus, Staphylococcal (Staph), Vibrio Species Causing Vibriosis, and malaria parasite.

Statement 53. The methods of any preceding statement, wherein the liquid components of the compositions of any preceding statement are evaporated; thereby providing a dry active surface configured to provide photodynamic disinfectant properties.

Statement 54. The compositions of any preceding statement, wherein the photosensitizer is selected from the group consisting of methylene blue (CAS #61-73-4), Rose Bengal (CAS #632-69-9), Riboflavin (CAS #83-88-5), Toluidine Blue (CAS #92-31-9) and Eosin Blue (CAS #16423-68-0).

Statement 55. The compositions of any preceding statement, wherein the photosensitizer is selected from the group consisting of Curcumin, Verteporfin, Erythrosin B, New MB, and Eosin Y, Erythrosine.

Statement 56. The compositions of any preceding statement, wherein the photosensitizer is selected from the group consisting of PHOTOFRIM, Photochlor (CAS #149402-51-7), IR700 Chlorin e6, Protoporphyrin IX, NPe6PHCurcumin, Verteporfin, Erythrosin B, New MB, Eosin Y, and Erythrosine.

Statement 57. The compositions of any preceding statement, wherein the inclusion complex former is selected from the group consisting of cyclodextrins, unsubstituted cyclodextrins, alpha-cyciodextrin, beta-cyclodextrin, gamma-cyciodextrin, calixarenes, cryptands and crown ethers and derivatives of each of these.

Statement 58. The compositions of any preceding statement, wherein the inclusion complex former is covalently bonded to a nanoparticle.

Statement 59. An article or structure comprising an active photodynamic disinfectant surface, the surface comprising a component comprising the photosensitizer associated with the inclusion complex former.

Statement 60. The composition of statement 59, wherein the photosensitizer is selected from the group consisting of methylene blue (CAS #61-73-4), Rose Bengal (CAS #632-69-9), Riboflavin (CAS #83-88-5), Toluidine Blue (CAS #92-31-9) and Eosin Blue (CAS #16423-68-0).

Statement 61. The composition of statement 59, wherein the photosensitizer is selected from the group consisting of Curcumin, Verteporfin, Erythrosin B, New MB, and Eosin Y, Erythrosine.

Statement 62. The composition of statement 59, wherein the photosensitizer is selected from the group consisting of PHOTOFRIM, Photochlor (CAS #149402-51-7), IR700 Chlorin e6, Protoporphyrin IX, NPe6PHCurcumin, Verteporfin, Erythrosin B, New MB, Eosin Y, and Erythrosine.

Statement 63. The composition of statement 59, wherein the inclusion complex former is selected from the group consisting of cyclodextrins, unsubstituted cyclodextrins, alpha-cyclodextrin, beta-cyclodextrin, gamma-cyclodextrin, calixarenes, cryptands and crown ethers and derivatives of each of these.

Statement 64. The composition of statement 59, wherein the inclusion complex former is covalently bonded to a nanoparticle.

Statement 65. An article or structure selected from the group of liner board, liner, medium, corrugated, corrugated containers, boxes, corrugated boxes, sheet material, and corrugated sheet material comprising a dry active photodynamic disinfectant surface, the surface comprising a component comprising the photosensitizer associated with the inclusion complex former.

Statement 66. The composition of statement 65, wherein the photosensitizer is selected from the group consisting of methylene blue (CAS #61-73-4), Rose Bengal (CAS #632-69-9), Riboflavin (CAS #83-88-5), Toluidine Blue (CAS #92-31-9) and Eosin Blue (CAS #16423-68-0).

Statement 67. The composition of statement 65, wherein the photosensitizer is selected from the group consisting of Curcumin, Verteporfin, Erythrosin B, New MB, and Eosin Y, Erythrosine.

Statement 68. The composition of statement 65, wherein the photosensitizer is selected from the group consisting of PHOTOFRIM, Photochlor (CAS #149402-51-7), IR700 Chlorin e6, Protoporphyrin IX, NPe6PHCurcumin, Verteporfin, Erythrosin B, New MB, Eosin Y, and Erythrosine.

Statement 69. The composition of statement 65, wherein the inclusion complex former is selected from the group consisting of cyclodextrins, unsubstituted cyclodextrins, alpha-cyclodextrin, beta-cyclodextrin, gamma-cyclodextrin, calixarenes, cryptands and crown ethers and derivatives of each of these.

Statement 70. The composition of statement 65, wherein the inclusion complex former is covalently bonded to a nanoparticle.

Statement 71. The articles, methods or compositions of any preceding statement, comprising a nanoparticle, wherein the composition consisting of a photosensitizer associated with the inclusion complex former is bonded to the nanoparticle.

Statement 72. The articles, methods or compositions of any preceding statement, comprising a nanoparticle, wherein the composition consisting of a photosensitizer associated with the inclusion complex former is bonded to the nanoparticle and wherein the nanoparticle comprises PEG.

Statement 73. The articles, methods or compositions of any preceding statement, comprising a plurality of photosensitizer, where at least two of the photosensitizers have peak absorptions at different wavelengths.

Statement 74. The articles, methods or compositions of any of the other pending claims, wherein the surface, article or material is selected from the group consisting of a fiber (natural or synthetic), paper (paper products), plastics, woven fabric, non-woven fabric, fur, leather, a hard surface, glass surface, metal surface, stone surface, porous surfaces, a formed product, a composite material or web, a sheet of material, a roll of material, a mask, a gown, a coat, gloves, surfaces on a transportation device, a surface of a truck, a surface of a car, a surface of a plane, surface of a boat, a surface of a bus, clothing, PPE, masks, face protection, counter tops, tables, desks, seats, medical equipment surfaces, medical device surfaces, an x-ray machine surface, a CAT scanner surface, and an MRI surface.

Statement 75. A shipping container, selected from group consisting of cardboard boxes, paper boxes, plastic boxes, metal boxes, drums, tubes, and cartons having a PS on a surface.

Statement 76. A shipping material having PPR properties, the shipping container selected from the group consisting of boxes, tubes, containers, carboys, pouches, and bags, the shipping container comprising a structural material and a PS.

Statement 77. The shipping material of statement 76, wherein the structural material is selected from the group consisting of paper, cardboard, cellulosic materials, paper board, plastics, plastic materials, non-woven materials, fabrics, woven materials, paper materials, paper board materials, corrugated materials, metal materials, glass materials, and composites.

Statement 78. The shipping materials of statements 76 or 77, wherein the PS is methylene blue.

Statement 79. The method of forming a PPR shipping material comprising adding a PS into, on, or both, to the shipping material.

Statement 80. The method of statement 79, wherein the shipping material is selected from the group consisting of boxes, tubes, containers, carboys, pouches, and bags.

Statement 81. The method of forming a PPR structural material comprising adding a PS into, on, or both, to the structural material.

Statement 82. The method of statement 81, further comprising forming the PPR structural material into a shipping material.

Statement 83. The method of statement 82, wherein the shipping material is selected from the group comprising boxes, tubes, containers, carboys, pouches, and bags.

### HEADINGS AND EMBODIMENTS

It should be understood that the use of headings in this specification is for the purpose of clarity, and is not limiting in any way. Thus, the processes and disclosures described under a heading should be read in context with the entirely of this specification, including the various examples. The use of headings in this specification should not limit the scope of protection afford the present disclosures.

It is noted that there is no requirement to provide or address the theory underlying the novel and groundbreaking processes, materials, performance or other beneficial features and properties that are the subject of, or associated with, embodiments of the present disclosures. Nevertheless, various theories are provided in this specification to further advance the art in this area. The theories put forth in this specification, and unless expressly stated otherwise, in no way limit, restrict or narrow the scope of protection to be afforded the claimed disclosures. These theories many not be required or practiced to utilize the present disclosures. It is further understood that the present disclosures may lead to new, and heretofore unknown theories to explain the function-features of embodiments of the methods, articles, materials, devices and system of the present disclosures; and such later developed theories shall not limit the scope of protection afforded the present disclosures.

The various embodiments of systems, therapies, processes, compositions, applications, and materials set forth in this specification may be used for various other fields and for various other activities, uses and embodiments. Additionally, these embodiments, for example, may be used with: existing systems, therapies, processes, compositions, applications, and materials; may be used with systems, therapies, processes, compositions, applications, and materials that may be developed in the future; and with systems, therapies, processes, compositions, applications, and materials that may be modified, in-part, based on the teachings of this specification. Further, the various embodiments and examples set forth in this specification may be used with each other, in whole or in part, and in different and various combinations. Thus, for example, the configurations provided in the various embodiments of this specification may be used with each other. For example, the components of an embodiment having A, A′ and B and the components of an embodiment having A″, C and D can be used with each other in various combination, e.g., A, C, D, and A. A″ C and D, etc., in accordance with the teaching of this specification. The scope of protection afforded the present disclosures should not be limited to a particular embodiment, example, configuration or arrangement that is set forth in a particular embodiment, example, or in an embodiment in a particular figure.

The disclosure may be embodied in other forms than those specifically disclosed herein without departing from its spirit or essential characteristics. The described embodiments are to be considered in all respects only as illustrative and not restrictive.

What is claimed is:

1. A method comprising:
exposing one or more articles to a light source; and
applying an anti-microbial to the one or more articles, wherein the anti-microbial is configured to perform with a PS(ROS).

2. The method of claim 1, wherein the one or more articles are packaging material, plastic films, or mats.
